(19) United States
(12) Patent Application Publication
(10) Pub. No.: US 2009/0304679 A1
(43) Pub. Date: Dec. 10, 2009

(54) Antibodies as T cell receptor mimics, methods of production and uses thereof

(76) Inventor: Jon A. Weidanz, Abilene, TX (US)

Correspondence Address: DUNLAP CODDING, P.C., PO BOX 16370, OKLAHOMA CITY, OK, 73113, US

(21) Appl. No.: 12/380,605

(22) Filed: Feb. 27, 2009

Related U.S. Application Data
(63) Continuation-in-part of application No. 11/809,895, filed on Jun. 1, 2007.
Application No. 11/809,895 is a continuation-in-part of application No. 11/517,516, filed on Sep. 7, 2006.
Application No. 11/809,895 is a continuation-in-part of application No. 11/140,644, filed on May 27, 2005.
(60) Provisional application No. 61/067,328, filed on Feb. 27, 2008.
(60) Provisional application No. 61/191,871, filed on Sep. 12, 2008.
(60) Provisional application No. 60/810,079, filed on Jun. 1, 2006.
(60) Provisional application No. 60/838,276, filed on Aug. 17, 2006.
(60) Provisional application No. 60/752,737, filed on Dec. 20, 2005.
(60) Provisional application No. 60/714,621, filed on Sep. 7, 2005.
(60) Provisional application No. 60/574,857, filed on May 27, 2004.
(60) Provisional application No. 60/640,020, filed on Dec. 28, 2004.
(60) Provisional application No. 60/646,338, filed on Jan. 24, 2005.
(60) Provisional application No. 60/673,296, filed on Apr. 20, 2005.

(51) Int. Cl. A61K 39/395

(52) U.S. Cl. 424/130.1

(57) ABSTRACT

The presently disclosed and claimed invention relates to a methodology of producing and utilizing antibodies that recognize peptides associated with a tumorigenic or disease state, wherein the peptides are displayed in the context of HLA molecules. These antibodies may be utilized in therapeutic methods of mediating cell lysis.

### CROSS-REFERENCE TO RELATED APPLICATIONS

This application claims benefit under 35 U.S.C. 119(e) of U.S. Ser. No. 61/067,328, filed Feb. 27, 2008, and U.S. Ser. No. 61/191,871, filed Sep. 12, 2008. This application is also a continuation-in-part of U.S. Ser. No. 11/809,895, filed Jun. 1, 2007; which claims benefit under 35 U.S.C. 119(e) of U.S. Ser. No. 60/810,079, filed Jun. 1, 2006. Said application U.S. Ser. No. 11/809,895 is also a continuation-in-part of U.S. Ser. No. 11/517,516, filed Sep. 7, 2006; which claims benefit under 35 U.S.C. 119(e) of provisional applications U.S. Ser. No. 60/714,621, filed Sep. 7, 2005; U.S. Ser. No. 60/751,542, filed Dec. 19, 2005; U.S. Ser. No. 60/752,737, filed Dec. 20, 2005; and U.S. Ser. No. 60/838,276, filed Aug. 17, 2006. Said application U.S. Ser. No. 11/517,516 is also a continuation-in-part of U.S. Ser. No. 11/140,644, filed May 27, 2005; which claims benefit under 35 U.S.C. 119(e) of provisional applications U.S. Ser. No. 60/374,857, filed May 27, 2004; U.S. Ser. No. 60/640,020, filed Dec. 28, 2004; U.S. Ser. No. 60/646,338, filed Jan. 24, 2005; and U.S. Ser. No. 60/673,296, filed Apr. 20, 2005. The entire contents of each of the above-referenced applications are expressly incorporated herein by reference in their entirety.

### STATEMENT REGARDING FEDERALLY SPONSORED RESEARCH OR DEVELOPMENT

The government owns certain rights in the presently disclosed and claimed invention pursuant to a grant from the Advanced Technology Program of the National Institute of Standards and Technology (Grant #70NANB4H3048).

### BACKGROUND OF THE INVENTION

1. Field of the Invention

The presently disclosed and claimed invention relates generally to a methodology of utilizing antibodies that recognize peptides associated with a tumorigenic or disease state, wherein the peptides are displayed in the context of HLA molecules. These antibodies will mimic the specificity of a T cell receptor (TCR) such that the molecules may be used as therapeutic reagents.

2. Description of the Background Art

Class I major histocompatibility complex (MHC) molecules, designated HLA class I in humans, bind and display peptide antigen ligands upon the cell surface. The peptide antigen ligands presented by the class I MHC molecule are derived from either normal endogenous proteins (“self”) or foreign proteins (“nonself”) introduced into the cell. Nonself proteins may be products of malignant transformation or intracellular pathogens such as viruses. In this manner, class I MHC molecules convey information regarding the internal milieu of a cell to immune effector cells including but not limited to, CD8⁺ cytotoxic T lymphocytes (CTLs), which are activated upon interaction with “nonself” peptides, thereby lysing or killing the cell presenting such “nonself” peptides.

Class II MHC molecules, designated HLA class II in humans, also bind and display peptide antigen ligands upon the cell surface. Unlike class I MHC molecules which are expressed on virtually all nucleated cells, class II MHC molecules are normally confined to specialized cells, such as B lymphocytes, macrophages, dendritic cells, and other antigen presenting cells which take up foreign antigens from the extracellular fluid via an endocytic pathway. The peptides they bind and present are derived from extracellular foreign antigens, such as products of bacteria that multiply outside of cells, wherein such products include protein toxins secreted by the bacteria that often have deleterious and even lethal effects on the host (e.g., human). In this manner, class II molecules convey information regarding the fitness of the extracellular space in the vicinity of the cell displaying the class II molecule to immune effector cells, including but not limited to, CD4⁺ helper T cells, thereby helping to eliminate such pathogens. The extermination of such pathogens is accomplished by both helping B cells make antibodies against microbes, as well as toxins produced by such microbes, and by activating macrophages to destroy ingested microbes.

Class I and class II HLA molecules exhibit extensive polymorphism generated by systematic recombinatorial and point mutation events during cell differentiation and maturation resulting from allelic diversity of the parents; as such, hundreds of different HLA types exist throughout the world's population, resulting in a large immunological diversity. Such extensive HLA diversity throughout the population is the root cause of tissue or organ transplant rejection between individuals as well as of differing individual susceptibility and/or resistance to infectious diseases. HLA molecules also contribute significantly to autoimmunity and cancer.

Class I MHC molecules alert the immune response to disorders within host cells. Peptides which are derived from viral- and tumor-specific proteins within the cell are loaded into the class I molecule's antigen binding groove in the endoplasmic reticulum of the cell and subsequently carried to the cell surface. Once the class I MHC molecule and its loaded peptide ligand are on the cell surface, the class I molecule and its peptide ligand are accessible to cytotoxic T lymphocytes (CTL). CTLs survey the peptides presented by the class I molecule and destroy those cells harboring ligands derived from infectious or neoplastic agents within that cell.

While specific CTL targets have been identified, little is known about the breadth and nature of ligands presented on the surface of a diseased cell. From a basic scientific perspective, many outstanding questions remain in the art regarding peptide presentation. For instance, it has been demonstrated that a virus can preferentially block expression of HLA class I molecules from a given locus while leaving expression at other loci intact. Similarly, there are numerous reports of cancerous cells that downregulate the expression of class I HLA at particular loci. However, there is no data describing how (or if) the classical HLA class I loci differ in the peptides they bind. It is therefore unclear how class I molecules from the different loci vary in their interaction with viral- and tumor-derived ligands and the number of peptides each will present.

Discerning virus- and tumor-specific ligands for CTL recognition is an important component of vaccine design. Ligands unique to tumorigenic or infected cells can be tested and incorporated into vaccines designed to evoke a protective CTL response. Several methodologies are currently employed to identify potentially protective peptide ligands. One approach uses T cell lines or clones to screen for biologically active ligands among chromatographic fractions of eluted peptides (Cox et al., 1994). This approach has been employed to identify peptide ligands specific to cancerous cells. A second technique utilizes predictive algorithms to identify peptides capable of binding to a particular class I molecule based upon previously determined motif and/or individual ligand sequences (De Groot et al., 2001); however, there have been reports describing discrepancies between these algorithms and empirical data. Peptides having high predicted probability of binding from a pathogen of interest can then be synthesized and tested for T cell reactivity in various assays, such as but not limited to, precursor, tetramer and ELISpot assays.

Many cancer cells display tumor-specific peptide-HLA complexes derived from processing of inappropriately expressed or overexpressed proteins, called tumor associated antigens (TAAs) (Bernhard et al., 1996; Baxevanis et al., 2006; and Andersen et al., 2003). With the discovery of mAb technology, it was believed that “magic bullets” could be developed which specifically target malignant cells for destruction. Current strategies for the development of tumor specific antibodies rely on creating monoclonal antibodies (mAbs) to TAAs displayed as intact proteins on the surface of malignant cells. Though targeting surface tumor antigens has resulted in the development of several successful anti-tumor antibodies (Herceptin and Rituxan), a significant number of patients (up to 70%) are refractory to treatment with these antibody molecules. This has raised several questions regarding the rationale for targeting whole molecules displayed on the tumor cell surface for developing cancer therapeutic reagents. First, antibody-based therapies directed at surface antigens are often associated with lower than expected killing efficiency of tumor cells. Free tumor antigens shed from the surface of the tumor occupy the binding sites of the anti-tumor specific antibody, thereby reducing the number of active molecules and resulting in decreased tumor cell death. Second, current mAb molecules do not recognize many potential cancer antigens because these antigens are not expressed as an intact protein on the surface of tumor cells. The tumor suppressor protein p53 is a good example. p53 and similar intracellular tumor associated proteins are normally processed within the cell into peptides which are then presented in the context of either HLA class I or class II molecules on the surface of the tumor cell. Native antibodies are not generated against peptide-HLA complexes. Third, many of the antigens recognized by antibodies are heterogenic by nature, which limits the effectiveness of an antibody to a single tumor histology. For these reasons it is apparent that antibodies generated against surface expressed tumor antigens may not be optimal therapeutic targets for cancer immunotherapy.

The majority of proteins produced by a cell reside within intracellular compartments, thus preventing their direct recognition by antibody molecules. The abundance of intracellular proteins that is available for degradation by proteasome-dependent and independent mechanisms yields an enormous source of peptides for surface presentation in the context of the MHC class I system (Rock et al., 2004). A new class of antibodies that specifically recognizes HLA-restricted peptide targets (epitopes) on the surface of cancer cells would significantly expand the therapeutic repertoire if it could be shown that they have anti-tumor properties which could lead to tumor cell death.

Many T cell epitopes (specific peptide-HLA complexes) are common to a broad range of tumors which have originated from several distinct tissues. The primary goal of epitope discovery has been to identify peptide (tumor antigens) for use in the construction of vaccines that activate a clinically relevant cellular immune response against the tumor cells. The goal of vaccination in cancer immunotherapy is to elicit a cytotoxic T lymphocyte (CTL) response and activate T helper responses to eliminate the tumor. Although many of the epitopes discovered by current methods are immunogenic, shown by studies that generate peptide-specific CTL in vitro and in vivo, the application of vaccination protocols to cancer treatment has not been highly successful. This is especially true for cancer vaccines that target self-antigens (“normal” proteins that are overexpressed in the malignant cells). Although this class of antigens may not be ideal for vaccine formulation due to an individual “tolerance” of self antigens, they still represent good targets for eliciting antibodies ex vivo.

The value of monoclonal antibodies which recognize peptide-MHC complexes has been recognized by others (see for example Reiter, US Publication No. US 2004/0191260 A1, filed Mar. 26, 2003; Andersen et al., US Publication No. US 2002/0150914 A1, filed Sep. 19, 2001; Hoogenboom et al., US Publication No. US 2003/0223994 A1, filed Feb. 20, 2003; and Reiter et al., PCT Publication No. WO 03/068201 A2, filed Feb. 11, 2003). However, these processes employ the use of phage display libraries that do not produce a whole, ready-to-use antibody product. The majority of these antibodies were isolated from bacteriophage libraries as Fab fragments (Cohen et al., 2003; Held et al., 2004; and Chames et al., 2000) and have not been examined for anti-tumor activity since they do not activate innate immune mechanisms (e.g., complement-dependent cytotoxicity [CDC]) or antibody-dependent cellular cytotoxicity (ADCC). Demonstration of anti-tumor activity is critical, as therapeutic mAbs are thought to act through several mechanisms which engage the innate response, including antibody or complement-mediated phagocytosis by macrophage, CDC and ADCC (Liu et al., 2004; Prang et al., 2005; Akewanlop et al., 2001; Clynes et al., 2000; and Masui et al., 1986). These prior art methods also have not demonstrated production of antibodies capable of staining tumor cells in a robust manner, implying that they are of low affinity or specificity. The immunogen employed in the prior art methods uses MHC which has been “enriched” for one particular peptide, and therefore such immunogen contains a pool of peptide-MHC complexes and is not loaded solely with the peptide of interest. In addition, there has not been a concerted effort in these prior art methods to maintain the structure of the three dimensional epitope formed by the peptide/HLA complex, which is essential for generation of the appropriate antibody response. For these reasons, immunization protocols presented in these prior art references had to be carried out over long periods of time (i.e., approximately 5 months or longer).

In addition, the vast majority of phage-derived antibodies produced by the prior art methods will not fold right in mammalian cells due to their selection for expression in prokaryotic or simple eukaryotic systems; generally, <1% of phage-derived antibodies will efficiently fold in mammalian cells, thus greatly increasing the number of candidates that must be screened and virtually assuring that interesting lead candidates with the most desirable binding properties are non-producible in mammalian cells due to the infrequency of success. Supporting this contention is the fact that very few phage-derived antibodies have proceeded into clinical investigation, and no phage-derived antibody has been approved for use as a therapeutic. All approved therapeutic antibodies have their discovery origin from a mammalian species.

Thus, the prior art phage-derived antibodies are not useful for making anti-MHC/peptide complexes, as they typically exhibit low affinity, low robustness, low capability to grow and fold, and as they are generally laborious to implement and have not been shown to be viable for approved therapeutic use.

Therefore, there exists a need in the art for therapeutic antibodies with novel recognition specificity for peptide-HLA domain in complexes present on the surface of tumor or diseased/infected cells. The presently claimed and disclosed invention provides innovative processes for utilizing antibody molecules endowed with unique antigen recognition specificities for peptide-HLA complexes, as peptide-HLA molecules are unique sources of tumor/disease/infection specific antigens available as therapeutic targets.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates the expression of HLA/peptide complexes on MDA cells, as detected by T cell receptor mimics RL1B, RL4B and RL6A.

FIG. 2 graphically illustrates that TCRms RL4B and RL6A (A) or RL1B (B) increase tumor cell cytotoxicity.

FIG. 3 graphically illustrates that TCRms RL4B and RL6A (A) or RL1B (B) increase cell death on tumor cell lines.

FIG. 4 graphically depicts that TCRms RL4B and RL6A mediate CDC.

FIG. 5 graphically depicts that TCRm RL4B mediates ADCC of breast cancer cells.

FIG. 6 illustrates the in vivo efficacy of TCRm RL4B for cancer prophylaxis.

FIG. 7 depicts that TCRms RL6A (A) and RL4B (B) retard tumor growth in orthotopic breast cancer models.

FIG. 8 illustrates that TCRm RL6A debulks large tumors in orthotopic breast model in mice.

FIG. 9 depicts the West Nile Virus (WNV) genome map and peptide sequences.

FIG. 10 graphically illustrates titration of RL15A TCRm (anti-WNV3 peptide/HLA-A2 antibody).

FIG. 11 graphically depicts peptide titration with various concentrations of WNV-3 peptide and RL15ATCRm.

FIG. 12 graphically represents the examination of RL15A TCRm cross-reactivity with WNV peptides 1, 2, 4, 5 and 6.

FIG. 13 graphically represents that RL15A TCRm recognizes dengue type-1 peptide (DT1) peptide-HLA-A2 complex.

FIG. 14 graphically represents that RL15A TCRm recognizes dengue type-2 peptide (DT2) peptide-HLA-A2 complex.

FIG. 15 graphically represents that RL15A TCRm does not recognize dengue type-3 peptide (DT3) peptide-HLA-A2 complex.

FIG. 16 graphically represents that RL15A TCRm recognizes dengue type-4 peptide (DT4) peptide-HLA-A2 complex.

FIG. 17 graphically represents that RL15A TCRm recognizes Yellow Fever Virus (YFV) peptide (DT1) peptide-HLA-A2 complex.

FIG. 18 graphically represents that RL15A TCRm recognizes JEV/SEV peptide-HLA-A2 complex.

FIG. 19 graphically represents that RL15A TCRm recognizes Murray Valley Encephalitis Virus (MVEV) peptide-HLA-A2 complex.

FIG. 20 graphically depicts the examination of RL15A TCRm cross-reactivity for viral peptide-HLA-A2 epitopes.

FIG. 21 graphically illustrates the examination of RL15A TCRm cross-reactivity to cancer-associated peptide-HLA-A2 epitopes.

FIG. 22 graphically depicts titration of RL14C TCRm (anti-WNV6 peptide/HLA-A2 antibody).

FIG. 23 graphically illustrates peptide titration with WNV-6 peptide and RL14C TCRm.

FIG. 24 graphically depicts the examination of RL14C TCRm cross-reactivity with WNV peptides 1, 2, 3, 4 and 5.

FIG. 25 graphically illustrates the examination of RL14C TCRm cross-reactivity for viral peptide-HLA-A2 epitopes.

FIG. 26 graphically depicts the examination of RL14C TCRm cross-reactivity to cancer-associated peptide-HLA-A2 epitopes.

FIG. 27 illustrates an affinity determination of RL14C TCRm for cognate peptide-HLA-LA complex using BIAcore.

FIG. 28 demonstrates that TCRm RL15A specifically inhibits anti-SVG9/A2 CTL responses.

FIG. 29 illustrates that TCRm antibodies to WNV surface epitopes recognize naturally processed and presented peptide-HLA complexes.

FIG. 30 graphically illustrates inhibition of peptide-specific CTL lines using TCRm antibodies.

FIG. 31 demonstrates that DCs can cross-present HLA class-I restricted hCGβ epitopes to CD8⁺ T cells.

FIG. 32 illustrates that RL4D TCRm inhibits anti-GVL peptide-A2 reaction CTL after incubation with tumor cell lines.

### DETAILED DESCRIPTION OF THE INVENTION

Before explaining at least one embodiment of the invention in detail by way of exemplary drawings, experimentation, results, and laboratory procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and claimed invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2^{nd} ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The terms “isolated polynucleotide” and “isolated nucleic acid segment” as used herein shall mean a polynucleotide of genomic, cDNA, or synthetic origin or some combination thereof, which by virtue of its origin the “isolated polynucleotide” or “isolated nucleic acid segment” (1) is not associated with all or a portion of a polynucleotide in which the “isolated polynucleotide” or “isolated nucleic acid segment” is found in nature, (2) is operably linked to a polynucleotide which it is not linked to in nature, or (3) does not occur in nature as part of a larger sequence.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of murine proteins, (3) is expressed by a cell from a different species, or, (4) does not occur in nature.

The term “polypeptide” as used herein is a generic term to refer to native protein, fragments, or analogs of a polypeptide sequence. Hence, native protein, fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. In one embodiment, oligonucleotides are 10 to 60 bases in length, such as but not limited to, 12, 13, 14, 15, 16, 17, 18, 19, or 20 to 40 bases in length. Oligonucleotides are usually single stranded, e.g., for probes; although oligonucleotides may be double stranded, e.g., for use in the construction of a gene mutant. Oligonucleotides of the invention can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest will be at least 80%, and more typically with increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, such as at least 90 to 95 percent sequence identity, or at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-,α-disubstituted amino acids, N-alkyl amino acids, lactic acid, and other unconventional amino acids may also be suitable components for polypeptides of the presently disclosed and claimed invention. Examples of unconventional amino acids include: 4-hydroxyproline, ã-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

Similarly, unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, such as at least 90 percent sequence identity, or at least 95 percent sequence identity, or at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the presently disclosed and claimed invention, providing that the variations in the amino acid sequence maintain at least 75%, such as at least 80%, 90%, 95%, and 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (5) confer or modify other physicochemical or functional properties of such analogs. Analogs can include various mutations of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure ©. Branden and J. Tooze, eds., Garland Publishing, New York, N.Y. (1991)); and Thornton et at. Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, such as at least 14 amino acids long or at least 20 amino acids long, usually at least 50 amino acids long or at least 70 amino acids long.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

The term “MHC” as used herein will be understood to refer to the Major Histocompability Complex, which is defined as a set of gene loci specifying major histocompatibility antigens. The term “HLA” as used herein will be understood to refer to Human Leukocyte Antigens, which is defined as the histocompatibility antigens found in humans. As used herein, “HLA” is the human form of “MHC”.

The terms “MHC light chain” and “MHC heavy chain” as used herein will be understood to refer to portions of the MHC molecule. Structurally, class I molecules are heterodimers comprised of two noncovalently bound polypeptide chains, a larger “heavy” chain (a) and a smaller “light” chain (β-2-microglobulin or β2m). The polymorphic, polygenic heavy chain (45 kDa), encoded within the MHC on chromosome six, is subdivided into three extracellular domains (designated 1, 2, and 3), one intracellular domain, and one transmembrane domain. The two outermost extracellular domains, 1 and 2, together form the groove that binds antigenic peptide. Thus, interaction with the TCR occurs at this region of the protein. The 3 domain of the molecule contains the recognition site for the CD8 protein on the CTL; this interaction serves to stabilize the contact between the T cell and the APC. The invariant light chain (12 kDa), encoded outside the MHC on chromosome 15, consists of a single, extracellular polypeptide. The terms “MHC light chain”, “β-2-microglobulin”, and “β2m” may be used interchangeably herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is <1 μM, or <100 nM, or <10 nM.

The term “antibody” is used in the broadest sense, and specifically covers monoclonal antibodies (including full length monoclonal antibodies), polyclonal antibodies, multispecific antibodies (e.g., bispecific antibodies), and antibody fragments (e.g., Fab, F(ab′)₂ and Fv) so long as they exhibit the desired biological activity. Antibodies (Abs) and immunoglobulins (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond. While the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82 4592-4596 (1985).

An “isolated” antibody is one which has been identified and separated and/or recovered from a component of the environment in which it was produced. Contaminant components of its production environment are materials which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomasie blue or, preferably, silver stain. Isolated antibody includes the antibody in situ within recombinant cells since at least one component of the antibody's natural environment will not be present. Ordinarily, however, isolated antibody will be prepared by at least one purification step.

The term “antibody mutant” refers to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95%.

The term “variable” in the context of variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342: 877). The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat et al.) The constant domains are not involved directly in binding an antibody to an antigen, but exhibit various effector function, such as participation of the antibody in antibody-dependent cellular toxicity.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments which are capable of cross-linking antigen, and a residual other fragment (which is termed pFc′). As used herein, “functional fragment” with respect to antibodies, refers to Fv, F(ab) and F(ab′)₂ fragments.

An “Fv” fragment is the minimum antibody fragment which contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The Fab fragment [also designated as F(ab)] also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

The light chains of antibodies (immunoglobulin) from any vertebrate species can be assigned to one of two clearly distinct types, called kappa (κ) and lambda (λ), based on the amino sequences of their constant domain.

Depending on the amino acid sequences of the constant domain of their heavy chains, “immunoglobulins” can be assigned to different classes. There are at least five (5) major classes of immunoglobulins: IgA, IgD, IgE, IgG and IgM, and several of these may be further divided into subclasses (isotypes), e.g., IgG-1, IgG-2, IgG-3 and IgG4; IgA-1 and IgA-2. The heavy chains constant domains that correspond to the different classes of immunoglobulins are called α, Δ, ε, γ and μ, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed and claimed invention may be made by the hybridoma method first described by Kohler and Milstein, Nature 256, 495 (1975).

All monoclonal antibodies utilized in accordance with the presently disclosed and claimed invention will be either (1) the result of a deliberate immunization protocol, as described in more detail herein below; or (2) the result of an immune response that results in the production of antibodies naturally in the course of a disease or cancer. These monoclonal antibodies are distinguished from the prior art antibodies which are phage-derived, because said prior art phage-derived antibodies are not useful for making anti-MHC/peptide complexes, as they typically exhibit low affinity, low robustness, low capability to grow and fold, and as they are generally laborious to implement and have not been shown to be viable for approved therapeutic use.

Utilization of the monoclonal antibodies of the presently disclosed and claimed invention may require administration of such or similar monoclonal antibody to a subject, such as a human. However, when the monoclonal antibodies are produced in a non-human animal, such as a rodent, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed and claimed invention can be “humanized”, that is, the antibodies are engineered such that antigenic portions thereof are removed and like portions of a human antibody are substituted therefore, while the antibodies' affinity for specific peptide/MHC complexes is retained. This engineering may only involve a few amino acids, or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, the Specifications of which are all hereby expressly incorporated herein by reference in their entirety.

Humanized forms of antibodies are chimeric immunoglobulins, immunoglobulin chains or fragments thereof (such as Fv, Fab, Fab′, F(ab′)2 or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988; Verhoeyen et al., 1988), by substituting rodent CDRs or CDR sequences for the corresponding sequences of a human antibody. (See also U.S. Pat. No. 5,225,539.) In some instances, Fᵥ framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, 1992).

97 published articles relating to the generation or use of humanized antibodies were identified by a PubMed search of the database as of Apr. 25, 2002. Many of these studies teach useful examples of protocols that can be utilized with the presently disclosed and claimed invention, such as Sandborn et al., Gatroenterology, 120:1330 (2001); Mihara et al., Clin. Immunol. 98:319 (2001); Yenari et al., Neurol. Res. 23:72 (2001); Morales et al., Nucl. Med. Biol. 27:199 (2000); Richards et al., Cancer Res. 59:2096 (1999); Yenari et al., Exp. Neurol. 153:223 (1998); and Shinkura et al., Anticancer Res. 18:1217 (1998), all of which are expressly incorporated in their entirety by reference. For example, a treatment protocol that can be utilized in such a method includes a single dose, generally administered intravenously, of 10-20 mg of humanized mAb per kg (Sandborn, et al. 2001). In some cases, alternative dosing patterns may be appropriate, such as the use of three infusions, administered once every two weeks, of 800 to 1600 mg or even higher amounts of humanized mAb (Richards et al., 1999). However, it is to be understood that the invention is not limited to the treatment protocols described above, and other treatment protocols which are known to a person of ordinary skill in the art may be utilized in the methods of the presently disclosed and claimed invention.

The presently disclosed and claimed invention further includes the use of fully human monoclonal antibodies against specific peptide/MHC complexes. Fully human antibodies essentially relate to antibody molecules in which the entire sequence of both the light chain and the heavy chain, including the CDRs, arise from human genes. Such antibodies are termed “human antibodies” or “fully human antibodies” herein. Human monoclonal antibodies can be prepared by the trioma technique; the human B-cell hybridoma technique (see Kozbor, et al., Hybridoma, 2:7 (1983)) and the EBV hybridoma technique to produce human monoclonal antibodies (see Cole, et al., PNAS 82:859 (1985)). Human monoclonal antibodies may be utilized in the practice of the presently disclosed and claimed invention and may be produced by using human hybridomas (see Cote, et al., PNAS 80:2026 (1983)) or by transforming human B-cells with Epstein Barr Virus in vitro (see Cole, et al., 1985).

In addition, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example but not by way of limitation, in U.S. Pat. Nos. 5,545,807; 5,545,806; 5,569,825; 5,625,126; 5,633,425; 5,661,016, and in Marks et al., J Biol. Chem. 267:16007,(1992); Lonberg et al., Nature, 368:856 (1994); Morrison, 1994; Fishwild et al., Nature Biotechnol. 14:845 (1996); Neuberger, Nat. Biotechnol. 14:826 (1996); and Lonberg and Huszar, Int Rev Immunol. 13:65 (1995).

Human antibodies may additionally be produced using transgenic nonhuman animals which are modified so as to produce fully human antibodies rather than the animal's endogenous antibodies in response to challenge by an antigen. (See PCT publication WO 94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. One embodiment of such a nonhuman animal is a mouse, and is termed the XENOMOUSE™ as disclosed in PCT Publication Nos. WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598, issued to Kucherlapati et al. on Aug. 17, 1999, and incorporated herein by reference. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771, issued to Hori et al. on Jun. 29, 1999, and incorporated herein by reference. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

As used herein, the terms “label” or “labeled” refer to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, 35S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

The terms “label”, “detectable marker” and “detection moiety” are used interchangeably herein.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

As used herein, “substantially pure” means an object species is the predominant species present (i.e., on a molar basis it is more abundant than any other individual species in the composition). Generally, a substantially pure composition will comprise more than about 50% percent of all macromolecular species present in the composition, such as more than about 55%, 60%, 65%, 70%, 75%, 80%, 85%, 90%, 95%, and 99%. In one embodiment, the object species is purified to essential homogeneity (contaminant species cannot be detected in the composition by conventional detection methods) wherein the composition consists essentially of a single macromolecular species.

The term patient includes human and veterinary subjects.

A “liposome” is a small vesicle composed of various types of lipids, phospholipids and/or surfactant. The components of the liposome are commonly arranged in a bilayer formation, similar to the lipid arrangement of biological membranes.

“Treatment” refers to both therapeutic treatment and prophylactic or preventative measures. Those in need of treatment include those already with the disorder as well as those in which the disorder is to be prevented.

A “disorder” is any condition that would benefit from treatment with the polypeptide. This includes chronic and acute disorders or diseases including those pathological conditions which predispose the mammal to the disorder in question.

The terms “cancer” and “cancerous” refer to or describe the physiological condition in mammals that is typically characterized by unregulated cell growth. Examples of include but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer.

“Mammal” for purposes of treatment refers to any animal classified as a mammal, including human, domestic and farm animals, nonhuman primates, and zoo, sports, or pet animals, such as dogs, horses, cats, cows, etc.

As mentioned hereinabove, depending on the application and purpose, the T cell receptor mimic of the presently disclosed and claimed invention may be attached to any of various functional moieties. A T cell receptor mimic of the presently disclosed and claimed invention attached to a functional moiety may be referred to herein as an “immunoconjugate”. In one embodiment, the functional moiety is a detectable moiety or a therapeutic moiety.

As is described and demonstrated in further detail hereinbelow, a detectable moiety or a therapeutic moiety may be particularly employed in applications of the presently disclosed and claimed invention involving use of the T cell receptor mimic to detect the specific peptide/MHC complex, or to kill target cells and/or damage target tissues.

The presently disclosed and claimed invention include the T cell receptor mimics described herein attached to any of numerous types of detectable moieties, depending on the application and purpose. For applications involving detection of the specific peptide/MHC complex, the detectable moiety attached to the T cell receptor mimic may be a reporter moiety that enables specific detection of the specific peptide/MHC complex bound by the T cell receptor mimic of the presently disclosed and claimed invention.

While various types of reporter moieties may be utilized to detect the specific peptide/MHC complex, depending on the application and purpose, the reporter moiety may be a fluorophore, an enzyme or a radioisotope. Specific reporter moieties that may utilized in accordance with the presently disclosed and claimed invention include, but are not limited to, green fluorescent protein (GFP), alkaline phosphatase (AP), peroxidase, orange fluorescent protein (OFP), β-galactosidase, fluorescein isothiocyanate (FITC), phycoerythrin, Cy-chrome, rhodamine, blue fluorescent protein (BFP), Texas red, horseradish peroxidase (HPR), and the like.

A fluorophore may be employed as a detection moiety enabling detection of the specific peptide/MHC complex via any of numerous fluorescence detection methods. Depending on the application and purpose, such fluorescence detection methods include, but are not limited to, fluorescence activated flow cytometry (FACS), immunofluorescence confocal microscopy, fluorescence in-situ hybridization (FISH), fluorescence resonance energy transfer (FRET), and the like.

Various types of fluorophores, depending on the application and purpose, may be employed to detect the specific peptide/MHC complex. Examples of suitable fluorophores include, but are not limited to, phycoerythrin, fluorescein isothiocyanate (FITC), Cy-chrome, rhodamine, green fluorescent protein (GFP), blue fluorescent protein (BFP), Texas red, and the like.

Ample guidance regarding fluorophore selection, methods of linking fluorophores to various types of molecules, such as a T cell receptor mimic of the presently disclosed and claimed invention, and methods of using such conjugates to detect molecules which are capable of being specifically bound by antibodies or antibody fragments comprised in such immunoconjugates is available in the literature of the art [for example, refer to: Richard P. Haugland, “Molecular Probes: Handbook of Fluorescent Probes and Research Chemicals 1992-1994”, 5th ed., Molecular Probes, Inc. (1994); U.S. Pat. No. 6,037,137 to Oncoimmunin Inc.; Hermanson, “Bioconjugate Techniques,” Academic Press New York, N.Y. (1995); Kay M. et al., 1995. Biochemistry 34:293; Stubbs et al., 1996. Biochemistry 35:937; Gakamsky D. et al., “Evaluating Receptor Stoichiometry by Fluorescence Resonance Energy Transfer,” in “Receptors: A Practical Approach,” 2nd ed., Stanford C. and Horton R. (eds.), Oxford University Press, UK. (2001); U.S. Pat. No. 6,350,466 to Targesome, Inc.]. Therefore, no further description is considered necessary.

Alternately, an enzyme may be utilized as the detectable moiety to enable detection of the specific peptide/MHC complex via any of various enzyme-based detection methods. Examples of such methods include, but are not limited to, enzyme linked immunosorbent assay (ELISA; for example, to detect the specific peptide/MHC complex in a solution), enzyme-linked chemiluminescence assay (for example, to detect the complex on solubilized cells), and enzyme-linked immunohistochemical assay (for example, to detect the complex in a fixed tissue).

Numerous types of enzymes may be employed to detect the specific peptide/MHC complex, depending on the application and purpose. Examples of suitable enzymes include, but are not limited to, horseradish peroxidase (HPR), β-galactosidase, and alkaline phosphatase (AP). Ample guidance for practicing such enzyme-based detection methods is provided in the literature of the art (for example, refer to: Khatkhatay M I. and Desai M., 1999. J Immunoassay 20:151-83; Wisdom G B., 1994. Methods Mol Biol. 32:433-40; Ishikawa E. et al., 1983. J Immunoassay 4:209-327; Oellerich M., 1980. J Clin Chem Clin Biochem. 18:197-208; Schuurs A H. and van Weemen B K., 1980. J Immunoassay 1:229-49).

The presently disclosed and claimed invention includes the T cell receptor mimics described herein attached to any of numerous types of therapeutic moieties, depending on the application and purpose. Various types of therapeutic moieties that may be utilized in accordance with the presently disclosed and claimed invention include, but are not limited to, a cytotoxic moiety, a toxic moiety, a cytokine moiety, a bi-specific antibody moiety, and the like. Specific examples of therapeutic moieties that may be utilized in accordance with the presently disclosed and claimed invention include, but are not limited to, Pseudomonas exotoxin, Diptheria toxin, interleukin 2, CD3, CD16, interleukin 4, interleukin 10, Ricin A toxin, and the like.

The functional moiety may be attached to the T cell receptor mimic of the presently disclosed and claimed invention in various ways, depending on the context, application and purpose. A polypeptidic functional moiety, in particular a polypeptidic toxin, may be attached to the antibody or antibody fragment via standard recombinant techniques broadly practiced in the art (for Example, refer to Sambrook et al., infra, and associated references, listed in the Examples section which follows). A functional moiety may also be attached to the T cell receptor mimic of the presently disclosed and claimed invention using standard chemical synthesis techniques widely practiced in the art [for example, refer to the extensive guidelines provided by The American Chemical Society (for example at: http://www.chemistry.org/portal/Chemistry)]. One of ordinary skill in the art, such as a chemist, will possess the required expertise for suitably practicing such chemical synthesis techniques.

Alternatively, a functional moiety may be attached to the T cell receptor mimic by attaching an affinity tag-coupled T cell receptor mimic of the presently disclosed and claimed invention to the functional moiety conjugated to a specific ligand of the affinity tag. Various types of affinity tags may be employed to attach the T cell receptor mimic to the functional moiety. In one embodiment, the affinity tag is a biotin molecule or a streptavidin molecule. A biotin or streptavidin affinity tag can be used to optimally enable attachment of a streptavidin-conjugated or a biotin-conjugated functional moiety, respectively, to the T cell receptor mimic due to the capability of streptavidin and biotin to bind to each other with the highest non covalent binding affinity known to man (i.e., with a Kd of about 10⁻¹⁴ to 10⁻¹⁵).

A pharmaceutical composition of the presently disclosed and claimed invention includes a T cell receptor mimic of the presently disclosed and claimed invention and a therapeutic moiety conjugated thereto. The pharmaceutical composition of the presently disclosed and claimed invention may be an antineoplastic agent. A diagnostic composition of the presently disclosed and claimed invention includes a T cell receptor mimic of the presently disclosed and claimed invention and a detectable moiety conjugated thereto.

The presently disclosed and claimed invention relates to methodologies for utilizing an agent, such as but not limited to antibodies or antibody fragments that function as T-cell receptor mimics (TCRm's), that recognize peptides displayed in the context of HLA molecules, wherein the peptide is associated with a tumorigenic, infectious, disease or immune dysfunction state. These antibodies will mimic the specificity of a T cell receptor (TCR) such that the molecules may be used as therapeutic reagents. In one embodiment, the T cell receptor mimics of the presently disclosed and claimed invention will have a higher binding affinity than a T cell receptor. In one embodiment, the T cell receptor mimic produced by the method of the presently disclosed and claimed invention has a binding affinity of about 10 nanomolar or greater.

In one embodiment, the methods utilize a T-cell receptor mimic, as described in detail hereinabove and in U.S. Ser. No. 11/809,895, filed Jun. 1, 2007, and in US Published Application Nos. US 2006/0034850, filed May 27, 2005, and US 2007/00992530, filed Sep. 7, 2006, which have previously been incorporated herein by reference. The T-cell receptor mimic utilized in the methods of the presently disclosed and claimed invention comprises an antibody or antibody fragment reactive against a specific peptide/MHC complex, wherein the antibody or antibody fragment can differentiate the specific peptide/MHC complex from the MHC molecule alone, the specific peptide alone, and a complex of MHC and an irrelevant peptide. The T cell receptor mimic may be produced by any of the methods described in detail in the patent applications listed herein above and expressly incorporated herein by reference; for example but not by way of limitation, the T cell receptor mimic may be produced by immunizing a host with an effective amount of an immunogen comprising a multimer of two or more specific peptide/MHC complexes.

In one embodiment, the T cell receptor mimic utilized in accordance with the presently disclosed and claimed invention may be produced by a method that includes identifying a peptide of interest, wherein the peptide of interest is capable of being presented by an MHC molecule, and wherein the vaccine composition comprises the peptide of interest. An immunogen comprising a multimer of two or more peptide/MHC complexes is then formed, wherein the peptide of the peptide/MHC complex is the peptide of interest. An effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced, wherein the desired antibodies can differentiate the peptide/MHC complex from the MHC molecule alone, the peptide of interest alone, and a complex of MHC and irrelevant peptide. The desired antibodies are then isolated.

Table I provides a list of some of the peptides that have been utilized to produce TCRm's by the methods described in detail in U.S. Ser. No. 11/809,895, filed Jun. 1, 2007, and in US Published Application Nos. US 2006/0034850, filed May 27, 2005, and US 2007/00992530, filed Sep. 7, 2006, which have previously been incorporated herein by reference. The use of TCRm's produced using any of the peptides of SEQ ID NOS:1-97 is specifically contemplated by the presently disclosed and claimed invention. However, it is to be understood that the presently disclosed and claimed invention is not limited to TCRm's produced using said peptides, but rather the scope of the presently disclosed and claimed invention encompasses TCRm's raised against any specific peptide/MHC complex.

TABLE I
Peptides Utilized in the Methods of U.S. Ser.
Nos. 11/1140,644; 11/517,516; and 11/809,895

| | SEQ | |
|---|---|---|
| | ID | |
| Sequence | NO: | Origin |
| LLGRNSFEV | 8 | Tumor suppressor |
| | | p53 (264-272) |
| VLMTEDIKL | 9 | eukaryotic |
| | | transcription |
| | | initiation |
| | | factor 4 gamma |
| | | (720-728) |
| KIFGSLAFL | 5 | tyrosine kinase-type |
| | | cell surface receptor |
| | | Her2 (EC 2.7.1.112) |
| | | (C-erbB-2) (369-377) |
| TMTRVLQGV | 2 | human chorionic |
| | | gonadotropin-β |
| | | (40-48) |
| VLQGVLPAL | 3 | human chorionic |
| | | gonadotropin-β |
| | | (44-53) |
| GVLPALPQV | 4 | human chorionic |
| | | gonadotropin-β |
| | | (47-55) |
| YLLPAIVHI | 10 | p68 |
| TLAYLIFCL | 11 | CD 19 (296-304) |
| YLEPGPVT | 12 | GP100 (280-288) |
| SLLMWITQV | 13 | NY-ESO-1 (157-165) |
| ILAKFLHWL | 14 | Human telomerase |
| | | reverse transcriptase |
| | | (hTERT) (540-548) |
| GPRTAALGLL | 7 | Reticulocalbin |
| EVDPIGHLY | 6 | Mage-3 |
| AAGIGILTV | 15 | MART-1 (26-35) wild |
| | | type |
| AIMDKNIIL | 16 | |
| ALGIGILTV | 17 | MART-1 (26-35) |
| | | (27L) |
| ALMPVLNQV | 18 | Exosome Component |
| | | 6 (EXOSC6) (214- |
| | | 222) |
| ATDFKFAMY | 19 | G1/S-specific |
| | | cyclin-D2 |
| ATTNILEHY | 20 | TRP-2-6b |
| AVLPPLPQV | 21 | bLH (67-75) |
| EADPTGHSY | 22 | Mage-1 |
| ELTLGEFLKL | 23 | Survivin |
| FLAEDALIITV | 24 | H-RYK |
| FLSTLTIDGV | 25 | HLA-A*0201-RE from |
| | | endothelium |
| FLSELTQQL | 26 | Migration Inhibitory |
| | | Factor (MIF) |
| FLYDDNQRV | 27 | Topoisomerase |
| GILGFVFTL | 28 | Influenza MI |
| GLNEEIARV | 29 | HEC1 Kinetochore |
| | | associated 2 (330- |
| | | 338) |
| GVLPNIQAV | 30 | |
| GVYDGEEHSV | 31 | Mage-B2 |
| IADMGHLKY | 32 | Proliferating cell |
| | | nuclear antigen |
| ILDQKINEV | 33 | Ornithine |
| | | Decarboxylase, |
| | | ODC1 |
| ILKEPVHGV | 34 | HIV reverse |
| | | transcriptase |
| ILNSRPPSV- | 35 | Modified |
| OH | | |
| IMDQVPFSV | 36 | Gp100 (208-217) |
| | | (2M) |
| IPSIQSRGL | 37 | Influenza HA 339-347 |
| ITDQVPFSV | 38 | Gp100 (209-217) wild |
| | | type |
| ITNSRPPSV- | 39 | Native (wild type) |
| OH | | |
| KIFGALAFL | 40 | S5A |
| KIFGGLAFL | 41 | S5G |
| AEAMEVA | 1 | Influenza M1 (200- |
| | | 206) |
| AAEAMEVA | 83 | Influenza M1 (199- |
| | | 206) |
| LKNDLLENLQ | 84 | Influenza M1 (229- |
| | | 238) |
| LPFDKTTIM | 85 | Influenza NP (418- |
| | | 426) |
| LPFEKSTIM | 86 | Influenza NP (418- |
| | | 426) |
| SPIVPSFDM | 87 | Influenza NP (473- |
| | | 481) |
| NPIVPSFDM | 88 | Influenza NP (473- |
| | | 481) |
| NLVPMVVATV | 95 | CMV pp65 |
| SLLMWIQTV | 96 | NY-ESO-1 |
| KIFGKLAFL | 42 | S5K |
| KIGEGTYGV | 43 | Cyclin Dependent |
| | | Kinase 2 (CK2) (9- |
| | | 17) |
| KKLLTQHFVQEN | 44 | Mage-3 (157-170) |
| YLEY | | |
| KLGEGTYGV | 45 | |
| KLMSPKLYV | 46 | 19-(150-158) |
| KLQELNYNL | 47 | Stat1 |
| KVLEYVIKV | 48 | Mage-1 (278-286) |
| LKMESLNFI | 49 | 20-(147-155) |
| LPFDRTTVM | 50 | Influenza B7-2 (NP |
| | | 418-426) |
| NAITNAKII | 51 | RSV M |
| NLVPMVATV | 52 | CMV pp65 |
| QPEWFRNIL | 53 | Influenza PB1 |
| | | (418-426) |
| QPEWFRNVL | 54 | Influenza PB1 |
| | | (418-426) |
| RMFPNAPYL | 55 | Wilm's tumor gene |
| | | WT1 (126-134) |
| RPYSNVSNL | 56 | B7B2, set-binding |
| | | factor 1 |
| SIGGVFTSV | 57 | S(I)G9 |
| SLFLGILSV | 58 | 20-(188-196) |
| SLLMWITQC | 59 | HLA-A*0201-RE NY- |
| | | ESO-1 WT (157- |
| | | 165) |
| SLLEKREKT | 60 | HLA-A*0201-RE |
| | | from SP-17- |
| STAPPAHGV | 61 | MUC1 |
| STPPPGTRV | 62 | HLA-A*0201-RE |
| | | from p53 (149)- |
| SVGGVFTSV | 63 | West Nile Virus |
| | | SVG9 (430-438) |
| SYIGSINNI | 64 | HRSV M2-1 |
| TLHEYMLDL | 65 | HPV16 E7-1 |
| TLQDIVLHL | 66 | HPV18 E7-1 |
| TMMRVLQAV | 67 | bLH (60-68) |
| TPQSNRPVM | 68 | B7A9, RNA pol II |
| | | polypeptide A |
| VLQAVLPPL | 69 | bLH (64-72)- |
| VLQELNVTV | 70 | PR-1 (169-177) |
| VMAGVGSPYV | 71 | Her2-(773-782)- |
| YIFGSLAFL | 72 | |
| YKYKVVKIEPLGV | 73 | P46, 13 mer, HIV-1 |
| | | envelope |
| YLEPGPVTA | 74 | Gp100: 280-288 |
| | | Wild type |
| YLEPGPVTV | 75 | Gp10: 280-288 |
| | | (288V) |
| YLLEMLWRL | 76 | Epstein-Barr virus |
| | | (EBV) |
| YMLDLQPETT | 77 | HPV16 (E7₁₁₋₂₀) |
| RLDDDGNFQL | 78 | West Nile Virus |
| | | NS2b |
| ATWAENIQV | 79 | West Nile Virus |
| | | peptide ATW9-WNV |
| YTMDGEYRL | 80 | West Nile Virus N53 |
| | | YL9 |
| SLTSINVQA | 81 | West Nile Virus |
| | | peptide NS4b SA9 |
| SLFGQRIEV | 82 | West Nile Virus |
| | | peptide SLF9 (68- |
| | | 76) |
| SLGGVFTSI | 89 | DT2 |
| SVGGVLNSL | 90 | DT3 |
| SVGGLFTSL | 91 | DT4 |
| SIGGVFNSI | 92 | JEV/SEV |
| SVGGVFNSI | 93 | MVEV |
| SAGGFFTSV | 94 | YFV |
| YLEVGPVTA | 97 | gp100 |

However, the presently disclosed and claimed invention is to be understood to not be limited to the use of TCRm's. In addition to TCRm's, any agent capable of directly detecting peptide/MHC complexes on the surface of a cell and differentiating the peptide/MHC complex from the MHC molecule alone, the specific peptide alone and a complex of MHC and irrelevant peptide may be utilized in accordance with the presently disclosed and claimed invention. Examples of particular agents that may be utilized include, but are not limited to, soluble T-cell receptors, extracted T-cell receptors, antibodies, antibody fragments and the technologies described in any of the following US patents/publications: US Publication No. US 2006/0115470 A1, published on Jun. 1, 2006 and filed by Silence et al., on Nov. 7, 2003; US Publication No. US 2007/0178082 A1, published on Aug. 2, 2007 and filed by Silence et al., on Nov. 7, 2003; US Publication No. US 2006/0246477 A1, published on Nov. 2, 2006 and filed by Hermans et al., on Jan. 31, 2006; US Publication No. US 2006/0211088 A1, published on Sep. 21, 2006, and filed by Hermans et al., on Mar. 13, 2006; US Publication No. US 2005/0214857 A1, published on Sep. 29, 2005, and filed by Lasters et al., on Dec. 11, 2002; U.S. Pat. No. 6,818,418, issued to Lipovsek et al., on Nov. 16, 2004; U.S. Pat. No. 7,115,396, issued to Lipovsek et al., on Oct. 3, 2006; US Publication No. US 2005/0255548 A1, published on Nov. 17, 2005 and filed by Lipovsek et al., on Nov. 15, 2004; US Publication No. US 2007/0082365 A1, published on Apr. 12, 2007 and filed by Lipovsek et al., on Oct. 3, 2006; US Publication No. US 2006/0246059 A1, published on Nov. 2, 2006 and filed by Lipovsek et al., on Jul. 7, 2006; US Publication No. US 2006/0270604 A1, published on Nov. 30, 2006 and filed by Lipovsek et al., on Jul. 7, 2006; US Publication No. US 2008/0139791 A1, published on Jun. 12, 2008 and filed by Lipovsek et al., on Jun. 12, 2008; US Publication No. US 2006/0286603 A1, published on Dec. 21, 2006 and filed by Kolkman et al., on Mar. 28, 2006; US Publication No. US 2005/0053973 A1, published on Mar. 10, 2005 and filed by Kolkman et al, on May 5, 2004;US Publication No. US 2005/0089932 A1, published on April 28, 2005 and filed by Kolkman et al., on Jun. 17, 2004; US Publication No. US 2004/0175756 A1, published on Sep. 9, 2004 and filed by Kolkman et al., on Oct. 24, 2003; US Publication No. US 2005/0048512 A1, published on Mar. 3, 2005 and filed by Kolkman et al., on Oct. 24, 2003; US Publication No. US 2005/0221384 A1, published on Oct. 6, 2005 and filed by Kolkman et al., on Oct. 15, 2004; US Publication No. US 2006/0223114 A1, published on Oct. 5, 2006 and filed by Stemmer et al., on Nov. 16, 2005; US Publication No. US 2006/0234299 A1, published on Oct. 19, 2006 and filed by Stemmer et al., on Nov. 16, 2005; US Publication No. US 2008/0003611 A1, published on Jan. 3, 2008 and filed by Silverman et al., on Jul. 12, 2006;US Publication No. US 2006/0286066 A1, published on Dec. 21, 2006 and filed by Basran on Dec. 22, 2005; US Publication No. US 2006/0257406 A1, published on Nov. 16, 2006 and filed by Winter et al., on May 31, 2005; US Publication No. US 2006/0106203 A1, published on May 18, 2006 and filed by Winter et al., on Dec. 28, 2004; US Patent No. US 2006/0263768 A1, published on Nov. 23, 2006 and filed by Tomlinson et al, on Apr. 28, 2006; US Publication No. 2007/0065440 A1, published on Mar. 22, 2007 and filed by Tomlinson et al., on Apr. 10, 2006; U.S. Pat. No. 6,696,245, issued to Winter et al., on Feb. 24, 2004; US Publication No. US 2006/0280734 A1, published on Dec. 14, 2006 and filed by Winter et al., on Jun. 24, 2005; US Publication No. US 2006/0083747 A1, published on Apr. 20, 2006 and filed by Winter et al., on Jun. 24, 2005; US Publication No. US 2004/0202995 A1, published on Oct. 14, 2004 and filed by de Wildt et al., on Apr. 9, 2003; U.S. Pat. No. 7,235,641, issued Jun. 26, 2007 to Kufer et al.; US Publication No. US 2003/0148463 A1, published on Apr. 7, 2003 and filed by Kufer et al., on Dec. 19, 2002; U.S. Pat. No. 7,227,002, issued to Kufer et al., on Jun. 5, 2007; U.S. Pat. No. 7,323,440, issued to Zoeher et al., on Feb. 12, 2003; U.S. Pat. No. 6,723,538, issued to Mack et al., on Apr. 20, 2004; U.S. Pat. No. 7,112,324, issued to Dorken et al., on Sep. 26, 2006; U.S. Pat. No. 7,250,297, issued to Beste et al., on Jul. 31, 2007; U.S. Pat. No. 6,849,259, issued to Haurum et al., on Feb. 1, 2005; US Publication No. 2008/0131882 A1, published on Jun. 5, 2008 and filed by Rasmussen et al., on Jul. 20, 2005; U.S. Pat. No. 5,670,626, issued to Chang on Sep. 23, 1997; U.S. Pat. No. 5,872,222, and issued to Chang on Feb. 16, 1999. The contents of each of the above-referenced patents and patent applications are hereby expressly incorporated herein by reference in their entirety.

Other Examples of particular agents that may be utilized in accordance with the presently disclosed and claimed invention are described in detail in parent application U.S. Ser. No. 61/191/871, filed Sep. 12, 2008, the entire contents of which has been previously incorporated herein by reference.

The presently disclosed and claimed invention relates to a method of mediating lysis of tumorigenic cells expressing at least one specific peptide/MHC complex on a surface thereof, wherein the specific peptide of the at least one specific peptide/MHC complex is associated with a tumorigenic state. The term “tumorigenic cell” as used herein will be understood to refer to a cell showing aberrant growth or structural phenotype such that given time, that phenotype will not act cooperatively with normal body processes. The term “tumorigenic cell” as used herein will be understood to include not only fully-transformed tumor cells but also precancerous cells; that is, tumor associated antigens includes those associated with fully transformed cells as well as those associated with a precancerous state.

In the method, an agent is provided, wherein the agent comprises a composition reactive against a specific peptide/MHC complex; the agent can differentiate the specific peptide/MHC complex from the MHC molecule alone, the specific peptide alone, and a complex of MHC and an irrelevant peptide. The agent is contacted with tumorigenic cells expressing at least one specific peptide/MHC complex on a surface thereof, such that the agent mediates lysis of the tumor cells expressing the at least one specific peptide/MHC complex on a surface thereof.

In one embodiment, the agent is a T cell receptor mimic comprising an antibody or antibody fragment reactive against a specific peptide/MHC complex, wherein the T cell receptor mimic is produced by immunizing a host with an effective amount of an immunogen comprising a multimer of two or more specific peptide/MHC complexes.

The specific peptide may be associated with any cancer, including but not limited to, at least one of breast cancer, ovarian cancer, prostate cancer, lung cancer, multiple myeloma, biliary cancer, and pancreatic cancer. In one embodiment, the specific peptide is at least one of SEQ ID NOS:4, 5, 10, 18, 26, 29, 33 and 43.

In the method, the mechanism of mediation of cell lysis may comprise at least one of activation of complement-dependent cytotoxicity (CDC), activation of antibody-dependent cellular toxicity (ADCC), induction of apoptosis, and activation of an anti-proliferative effect.

The presently disclosed and claimed invention is also directed to a method of mediating lysis of infected cells expressing at least one specific peptide/MHC complex on a surface thereof, wherein the specific peptide of the at least one specific peptide/MHC complex is associated with an infectious disease state. In the method, an agent is provided, wherein the agent comprises a composition reactive against a specific peptide/MHC complex, wherein the agent can differentiate the specific peptide/MHC complex from the MHC molecule alone, the specific peptide alone, and a complex of MHC and an irrelevant peptide. The agent is then contacted with infected cells expressing at least one specific peptide/MHC complex on a surface thereof, such that the agent mediates lysis of the infected cells expressing the at least one specific peptide/MHC complex on a surface thereof.

In one embodiment, the agent is a T cell receptor mimic comprising an antibody or antibody fragment reactive against a specific peptide/MHC complex, wherein the T cell receptor mimic is produced by immunizing a host with an effective amount of an immunogen comprising a multimer of two or more specific peptide/MHC complexes.

In one embodiment, the specific peptide may be associated with a bacterial infection. That is, the peptide may have been identified as being expressed in complex with a MHC molecule on the surface of a bacterially-infected cell and not expressed on a surface of a normal, non-infected cell.

In another embodiment, the specific peptide may be associated with a viral infection. That is, the peptide may have been identified as being expressed in complex with a MHC molecule on the surface of a virally-infected cell and not expressed on a surface of a normal, non-infected cell. Examples include, but are not limited to, specific peptides associated with an HIV infection, such as but not limited to, SEQ ID NOS:37 and 43; a flavivirus, including but not limited to, West Nile virus (such as but not limited to, SEQ ID NOS:63 and 78-82), and influenza virus (such as but not limited to, SEQ ID NOS:1, 28, 37, 50, 53, 54 and 83-88); hepatitis B; hepatitis C; human papilloma virus (HPV); herpes virus; cytomegalovirus (CMV) and Epstein-Barr virus (EBV).

In the method, the mechanism of mediation of cell lysis may comprise at least one of activation of complement-dependent cytotoxicity (CDC), activation of antibody-dependent cellular toxicity (ADCC), induction of apoptosis, and activation of an anti-proliferative effect.

The presently disclosed and claimed invention is also directed to a method of blocking autoreactive T cells activated by cells expressing at least one specific peptide/MHC complex on a surface thereof, wherein the specific peptide of the at least one specific peptide/MHC complex is associated with an autoimmune state. In the method, an agent is provided, wherein the agent comprises a composition reactive against a specific peptide/MHC complex, wherein the agent can differentiate the specific peptide/MHC complex from the MHC molecule alone, the specific peptide alone, and a complex of MHC and an irrelevant peptide. The agent is then contacted with cells expressing at least one specific peptide/MHC complex on a surface thereof, such that the agent binds to the surface of the cell and blocks binding and activation of autoreactive T cells.

In one embodiment, the agent is a T cell receptor mimic comprising an antibody or antibody fragment reactive against a specific peptide/MHC complex, wherein the T cell receptor mimic is produced by immunizing a host with an effective amount of an immunogen comprising a multimer of two or more specific peptide/MHC complexes.

The specific peptide may be any peptide associated with an autoimmune state, including but not limited to, at least one of SEQ ID NOS:2 and 4.

In one embodiment, the T cell receptor mimic may have at least one functional moiety, such as but not limited to, a detectable moiety or a therapeutic moiety, bound thereto. For example but not by way of limitation, the detectable moiety may be selected from the group consisting of a fluorophore, an enzyme, a radioisotope and combinations thereof, while the therapeutic moiety may be selected from the group consisting of a cytotoxic moiety, a toxic moiety, a cytokine moiety, a bi-specific antibody moiety, and combinations thereof.

The presently disclosed and claimed invention is also related to a method of killing or damaging a target cell expressing or displaying an antigen-presenting portion of a complex composed of a human antigen-presenting molecule and an antigen derived from a pathogen. The method involves exposing the target cell to a T cell receptor mimic as defined herein above, thereby killing or damaging a target cell expressing or displaying an antigen-presenting portion of a complex composed of a human antigen-presenting molecule and an antigen derived from a pathogen.

Examples are provided hereinbelow. However, the presently disclosed and claimed invention is to be understood to not be limited in its application to the specific experimentation, results and laboratory procedures. Rather, the Examples are simply provided as one of various embodiments and are meant to be exemplary, not exhaustive.

### Example 1

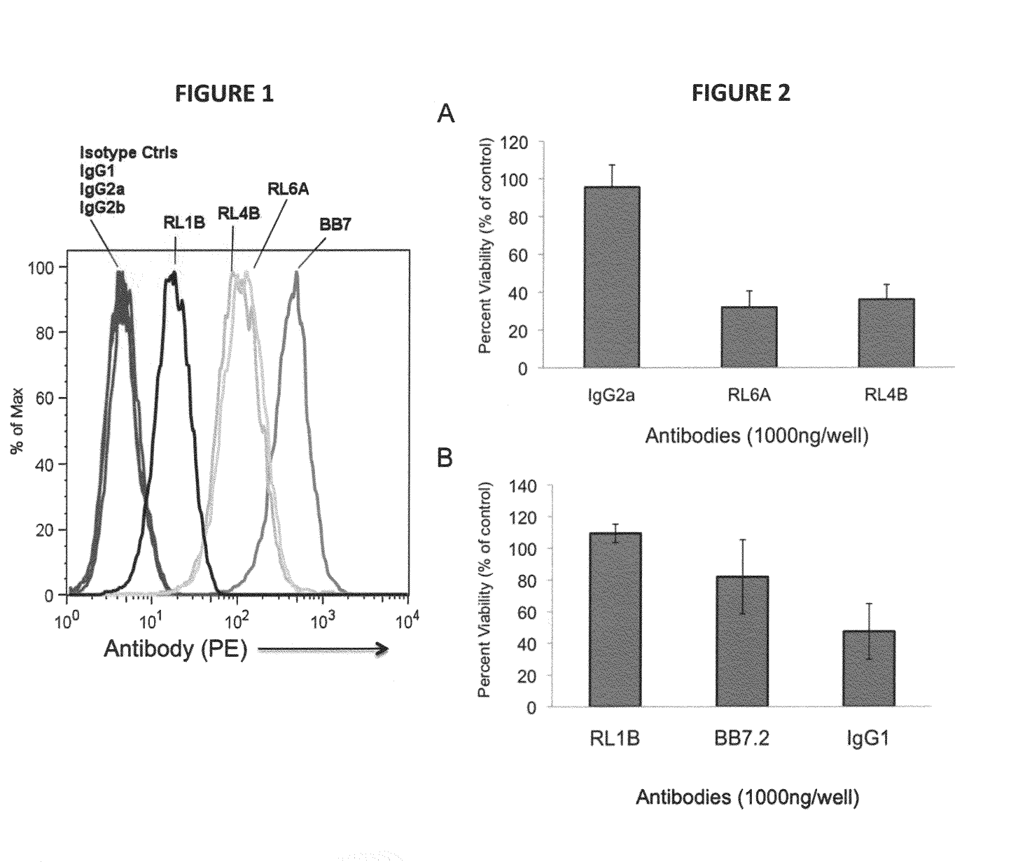

Detection of endogenously processed and presented peptide/HLA-A2 epitopes using RL1B, RL4B and RL6A TCRms. The inventors have previously observed that the RL1B, RL4B and RL6A TCR mimic monoclonal antibodies (TCRms) recognize recombinant HLA-A2 protein or T2 cells pulsed with the Her2/neu p369 peptide (SEQ ID NO:5), hCGb p47 peptide (SEQ ID NO:4) or the RNA helicase p68 peptide (SEQ ID NO:10), respectively. Next, it was evaluated whether these antibodies recognized cognate peptide/HLA-A2 complex on the surface of MDA-MB-231 tumor cells. Cells were stained with 0.5 μg of IgG1, IgG2A and IgG2b isotype control mAbs, RL1B, RL4B, RL6A TCRms and the pan-HLA-A2 antibody BB7.2. As shown in FIG. 1, RL4B and RL6A TCRm mAbs stained cells with significantly stronger intensity than cells stained with the RL1B TCRm suggesting that the Her2/neu peptide/HLA-A2 epitope is present on MDA-MB-231 tumor cells at a lower copy number than the epitopes recognized by RL4B and RL6A. Brightest staining was observed using BB7.2 mAb, and no cell staining was seen with any of the isotype control antibodies. Overall these results indicate that TCRm mAbs can be used in the detection and validation of epitopes which are endogenously processed and presented on the surface of tumor cells.

In vitro cytotoxic assay. To begin to evaluate the anti-tumor activities of TCRm, the cytotoxic properties of these agents was investigated in vitro. In the first of two studies, TCRm antibodies RL4B and RL6A were incubated with MDA-MB-231 tumor cells and assessed for cytotoxic effects using an MTT cell viability assay. The MTT assay measures cell viability through assessing mitochondrial reductase enzyme activity. The results from this study are shown in FIG. 2A and reveal that both RL4B and RL6A TCRm reduce tumor cell viability. In particular, both RL4B and RL6A used at 500 ng/ml reduced tumor cell viability by almost 70% after incubation together for 24 hrs and the isotype control antibody had little if any cytotoxic effect on the tumor cells In FIG. 2B, results of RL1B TCRm on MDA-MB-231 tumor cell viability after 24 h incubation were shown. Similar to the findings observed with RL4B and RL6A, RL1B used at 500 ng/ml reduced tumor cell viability to <50% while the pan-HLA-A2 mAb, BB7.2 reduced viability by 20%. These findings demonstrate a novel mechanism mediated by TCRm that reduces tumor cell viability. Other groups have reported cytotoxic effects of anti-HLA and anti-β2 microglobulin antibodies on a variety of normal and malignant cell types (Pathway of apoptosis induced in Jurkat T lymphoblasts by anti-HLA class I antibodies. Daniel D, Opelz G, Mulder A, Kleist C, Susal C. Hum Immunol. March 2004; 65(3):189-99, Fas-independent apoptosis of activated T cells induced by antibodies to the HLA class I alpha1 domain. Genestier L, Paillot R, Bonnefoy-Berard N, Meffre G, Flacher M, Fèvre D, Liu Y J, Le Bouteiller P, Waldmann H, Engelhard V H, Banchereau J, Revillard J P. Blood. Nov. 1, 1997; 90(9):3629-39). Although the negative cellular effects caused by HLA complex signaling have been reported previously, the inability of these antibodies to selectively target tumor cells and not normal cells limits their therapeutic usefulness. In contrast, targeting diseased cells using TCRm agents having specific recognition selectivity ultimately represents a unique therapeutic agent. The overall concept of TCRm docking onto cognate peptide/HLA-A2 and triggering a potent intracellular signaling event that can lead to a reduction in cell viability is novel and represents a potentially useful treatment modality for cancer.

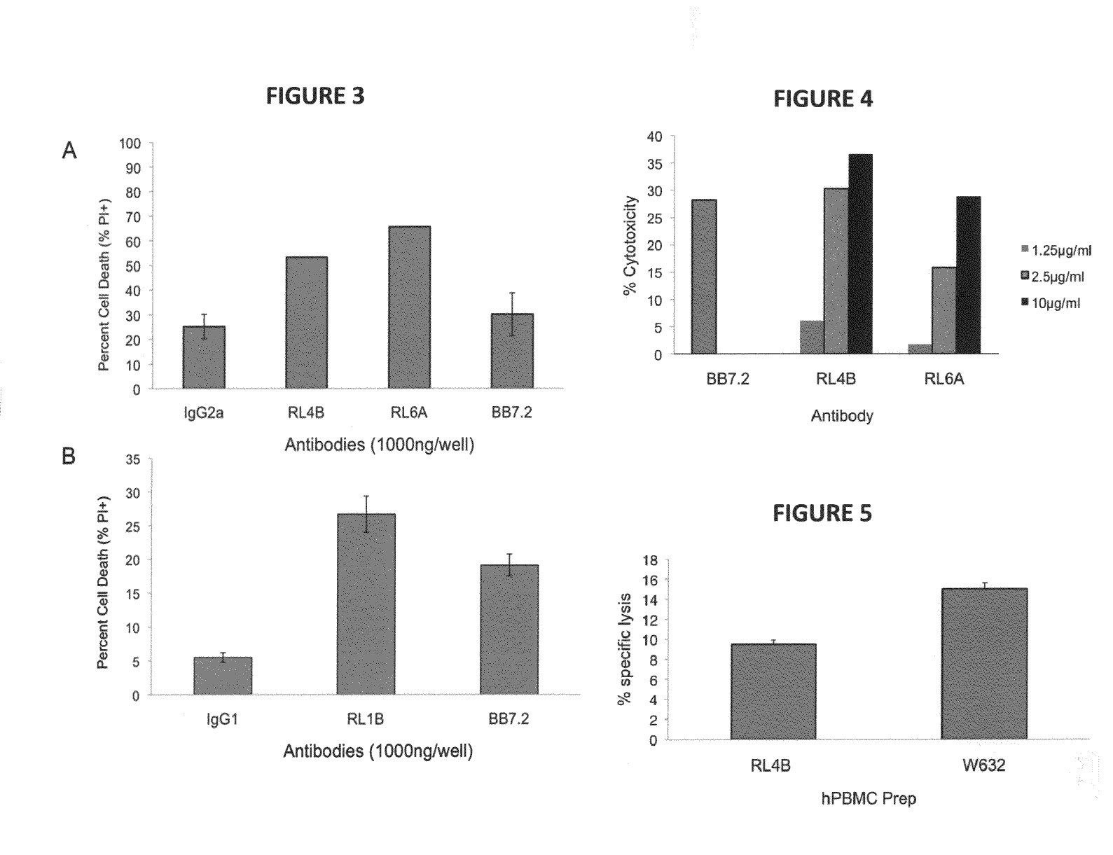

TCRm antibodies induce tumor cell death. To determine whether TCRm engagement of HLA/peptide complexes induces tumor cell death, uptake of Annexin-V and propidium iodide (PI) was assessed. MDA-MB-231 tumor cells were grown for 24 h in the absence or presence of isotype control antibody, BB7.2, RL4B TCRm or RL6A TCRm. Cell death was evaluated after 24 h incubation using Annexin-V tagged with APC fluorophore and propridium iodide (PI) by flow cytometric analysis. Shown in FIG. 3A, the percent of dead and/or dying cells are plotted after treatment with respected antibody or TCRm. The BB7.2 mAb was found to induce cell death that was approximately 2-fold greater than the isotype control antibody while both RL4B and RL6A TCRms induced far greater cell death at almost 3-fold more than the isotype control. FIG. 3B shows the results of RL1B TCRm mediated tumor cell death. As seen in this figure, BB7.2 induced approximately 3-fold greater cell death than the isotype control while the RL1B TCRm generated tumor cell death that was >5-fold higher than the isotype control antibody. Together, these finding indicate the ability of TCRm antibodies to selectively bind and induce cell death of tumor cells and thus represent a novel killing mechanism for these agents.

RL4B and RL6A TCRm mAbs direct complement dependent cytotoxic (CDC) killing of a human tumor cell line in vitro. The breast cancer cell line MDA-MB-231 was subjected to TCRm mediated CDC in the same manner in which the T2 cells were evaluated (data not shown). Tumor cells were plated and allowed to adhere overnight before antibody was applied. Antibody concentration was varied from 10 to 1.25 μg/ml. Murine IgG2a antibodies have been found to efficiently direct complement dependent cytolysis (CDC) while the IgG1 isotype does not. This fact and the corresponding ability of the IgG2a isotope to bind human Fc receptors led to our selection of the RL4B and RL6A TCRm mAbs and not RL1B in CDC assays. T2 cells pulsed with various peptides were used as targets for the initial RL4B and RL6A-directed CDC analysis because they could easily be loaded to a high density with any of a number of peptides (data not shown). FIG. 4 illustrates the CDC results of MDA-MB-231 tumor cells for the HLA-A2 specific BB7.2 antibody, RL4B and RL6A TCRms. CDC of cells incubated with antibody showed an antibody concentration-dependent lysis using RL4B and RL6A TCRms. Lysis was not seen for cells treated with isotype control antibody. This experiment implies that TCRm antibodies are potent activators of complement resulting in tumor cell lysis and offers a novel approach for targeting and killing tumor cells.

RL4B TCRm mediates antibody dependent cell-mediated cytotoxicity (ADCC) killing of tumor cells in vitro. Another mechanism which plays an important role in the ability of a therapeutic antibody to control or eliminate tumors is antibody-dependent cell-mediated cytotoxicity (ADCC). In order to investigate the ability of the RL4B TCRm to direct ADCC, peripheral blood mononuclear cells were isolated from the platelet chambers of aphaeresis collection devices from anonymous donors. The cells were held in serum-free medium (AIM-V) containing 200 units/ml rhIL-2 for 2 to 7 days with media changes every 2 to 3 days in order to maintain and activate the NK population. To determine the level of NK activity present in the different donor samples, each preparation was evaluated using the NK-sensitive cell line K562 at the same time the ADCC assays were carried out. All PBMC isolates were shown to exhibit lysis levels of 60% or more with one exception (35%) (data not shown).

MDA-MB-231 cells were first evaluated for sensitivity to ADCC as adherent cultures using five different human PBMC preparations to control for variation among the individual donors. FIG. 5 shows the results of these assays, which contained 10 μg/ml of RL4B TCRm and were run at an effector cell to target cell ratio (E:T) of 30:1. The PBMC preparations varied in their ability to lyse MDA cells as might be anticipated due to differences in receptor expression by NK cells. The overall ADCC ranged from 6.8 to 9.6% with an average value of 8.7% after subtraction and normalization of IgG2a isotype control.

To determine the effect epitope density had on overall lysis, RL4B TCRm or the pan-HLA antibody W6/32, which is also a murine isotype IgG2a, were used as targeting agents. The results from an ADCC analysis of MDA-231 cells using two different human donor preparations at an E:T ratio of 20:1 with RL4B and W6/32. The lysis values achieved for W6/32 (14.6-22.6%) were greater than those of RL4B (6.4-13.4%) suggesting that lysis was at least in part dependent on epitope density. Overall, these results show a modest but consistent level of tumor-specific ADCC mediated by the RL4B TCRm.

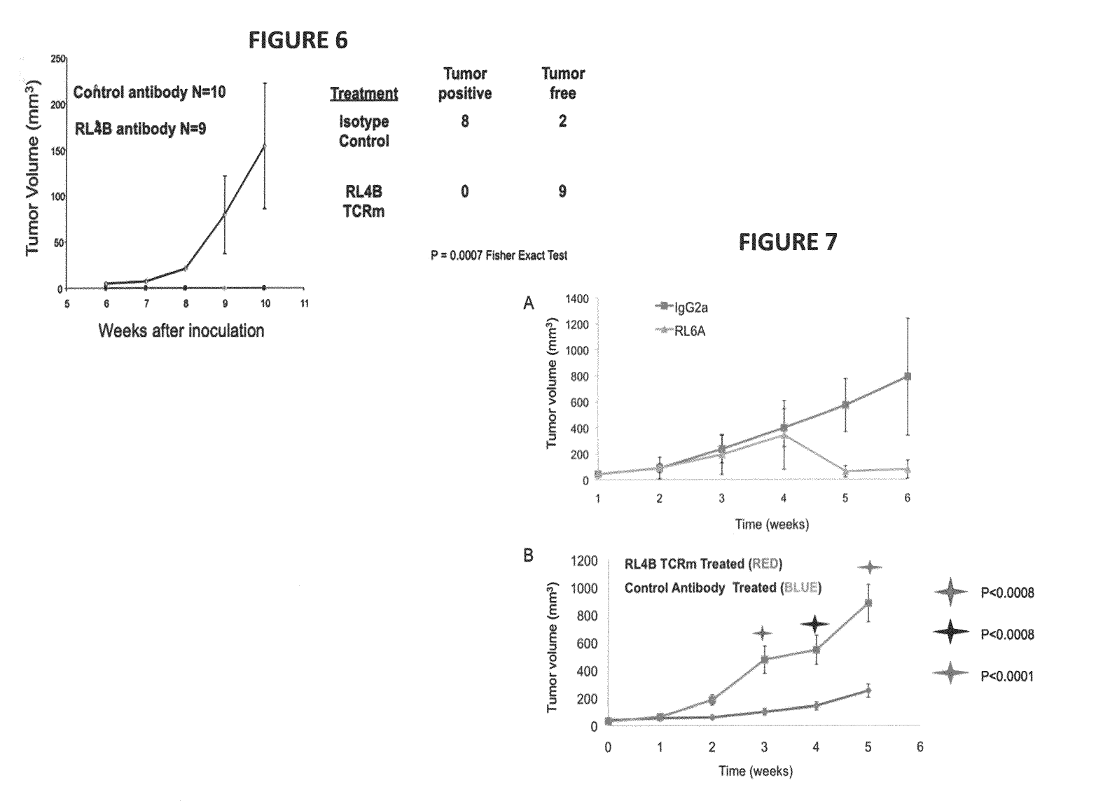

In vivo analysis of RL4B TCRm anti-tumor activity in nude mice implanted with MDA-MB-231 tumor cells. To establish the ability of the RL4B TCRm to inhibit tumor growth in vivo, nude mice were implanted with MDA-MB-231 tumor cells. Antibody treatment was initiated at the time of implantation with an intra-peritoneal (i.p.) injection of either RL4B TCRm or an isotype control antibody at a dose of 1.5 mg/kg. Tumors began to appear in the isotype control-treated mice between 36 and 43 days (week 6) after implantation, while none were evident in any of the mice treated with RL4B. Tumors continued to appear and expand in the control mice until day 69 (week 6 tumor volume˜4.5 mm³; week 10 tumor volume˜156 mm³). Final scoring was tabulated on day 69, 21 days after the appearance of the last tumor in the control mice. At day 69, eight of ten mice in the isotype treated group had developed tumors that were 6 mm in diameter or larger while none of the nine mice in the group treated with the RL4B TCRm showed evidence of tumor growth (FIG. 6). The experiment was terminated at 71 days.

TCRms prevent tumor growth in breast cancer orthotopic model. To further evaluate the anti-tumor properties of TCRm in vivo, athymic nude mice were implanted in the right mammary fat pads with a formulation mixture that was comprised of 5×10⁶ MDA-MB-231 tumor cells and Matrigel. Matrigel was used to create a nutrient rich environment that led to rapid tumor growth resulting in palpable tumors in 4 to 5 weeks after cell implantation. Tumors were allowed to grow to a mean volume of approximately ≧35 mm³ prior to initiation of treatment with RL4B or isotype control antibody (FIG. 7A) and RL6A or isotype control antibody (FIG. 7B). Mice were treated with weekly injections of either TCRm or control antibody at a concentration of 1.5 mg/kg. Mice that received RL4B (n=18) showed retarded tumor growth at 5 weeks after treatment (week 0 tumor volume˜35 mm³; week 5 tumor volume˜200 mm³). In contrast isotype control treated mice (n=15) showed rapid tumor growth with a mean tumor volume of >800 mm³ after 5 weeks of treatment, as shown in FIG. 7A.

Next, athymic nude mice implanted with MDA-MB-231 tumor cells in Matrigel were treated with RL6A TCRm (FIG. 7B). In this example one group of mice (n=4) received 5-weekly injections of isotype control antibody at 1.5 mg/kg. Tumor volume at week 0 was ≧35 mm³ and grew to a mean tumor volume of ˜800 mm³ by week 5. In contrast, the mean tumor volume in athymic nude mice treated with RL6A (n=3) at a dose of 1.5 mg/kg initially grew but mean tumor size was reduced by the fourth week and not even palpable after the fifth week after RL6A treatment. Interestingly, no palpable tumors were detected in the RL6A treated mice for an additional 4 weeks (data not shown). Collectively, these findings demonstrate the therapeutic effects of TCRm antibodies in preventing, controlling and/or reducing tumor growth.

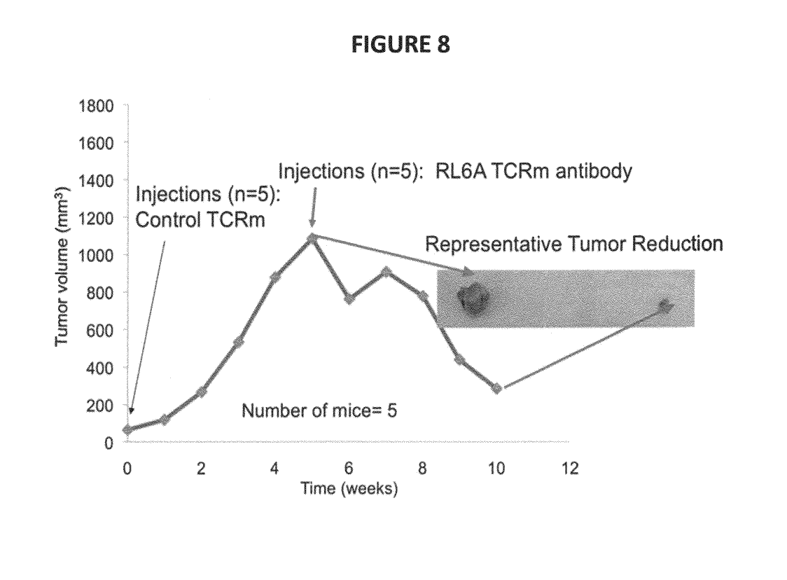

TCRm are useful reagents for debulking large established tumors. A significant test at demonstrating the anti-tumor properties of an antibody in vivo is for the antibody to shrink or debulk large established tumors in mice. Shown in FIG. 8 are results from treatment of athymic nude mice (n=5) having large human breast tumors. Tumors were established from MDA-MB-231 cancer cells after implantation into the right mammary fat pads. Mice received weekly injections of isotype control antibody (15 mg/kg) for 5 weeks without any impact on tumor growth retardation (mean tumor volume grew from ≧35 mm³ to ≧1,000 mm³). At peak mean tumor volume (>1000 mm³), mice received weekly i.p. injections of RL6A TCRm (15 mg/kg) for 5 weeks. By week 10 the mean tumor volume had decreased to <300 mm³.

These findings demonstrate that TCRm's have potent anti-tumor activity in vivo and support the uses of TCRm's as agents for tumor shrinkage.

### Materials and Methods for Example 1

Primary cells, cell lines and antibodies. The human tumor cell line MDA-MB-231(breast) was obtained from the American Type Culture Collection (ATCC). The murine IgG2a isotype control Abs was purchased from Sigma-Aldrich. Fresh blood buffy coats containing peripheral blood mononuclear cells were obtained from anonymous blood donations from Coffee Memorial Blood Bank (Amarillo, Tex.).

Antibodies and synthetic peptides. Polyclonal antibody goat anti-mouse IgG heavy chain-phycoerythrin (PE) was purchased from Caltag Laboratories (Burlingame, Calif.). Isotype control antibodies, mouse IgG1, IgG2a and IgG2b, were purchased from Southern Biotech (Birmingham, Ala.). The BB7.2 anti-HLA A2.1 mAb expressing mouse hybridoma cell line was purchased from the ATCC. Peptides, KIFGSLAFL (residues 369-377, designated as Her-2369; SEQ ID NO:5), RNA Helicase p68 YLLPAIVHI (residues 720-728, designated as p68; SEQ ID NO:10) and human chorionic gonadotropin-β GVLPALPQV (residues 47-55, designated as GVL47; SEQ ID NO:4) were synthesized at the University of Oklahoma Health Science Center, Oklahoma City, Okla., using a solid-phase method and purified by HPLC to greater than 90%.

Cell culture. Cell culture medium included IMDM from Cambrex. Medium supplements included heat-inactivated FBS and penicillin/streptomycin from Sigma-Aldrich and L-glutamine from HyClone. All tumor lines were maintained in culture medium containing glutamine, penicillin/streptomycin and 10% FBS. When necessary, attached cells were released from flasks using TrypLE express (Invitrogen Life technologies).

Tumor Cell staining. Tumor cells (3×10⁵) were washed and resuspended in (0.1 ml) FACS buffer followed by primary incubation with either 500 ng/stain of RL4B or RL6A TCRmimic antibody, 1000 ng/stain of RL1B TCRmimic antibody, 1000 ng/stain isotype controls (IgG1, IgG2a, IgG2b) or 1000 ng/stain of the anti-HLA-A2 antibody (BB7.2a). Cells were stained for 40 minutes protected from the light followed by addition of 2 ml FACS buffer to each tube and at 1100 RPM for 10 minutes. Cells were resuspended in 100 μl of FACS buffer. Secondary antibody incubation was done with goat anti-mouse secondary antibody (FITC or PE labeled) for 15 minutes protected from the light. After incubation the cells were washed and resuspended in FACS buffer. Cells were analyzed on FACS Scan (BD Biosciences) and analyzed using FlowJo analysis software (Tree Star Inc., Ashland, Oreg.).

In vitro studies.

a) Cytotoxicity studies. MDA-MB231 cells were plated on a 96 well plate at a density of 10,000 cells/well in cell culture medium and allowed to adhere overnight in an incubator at 37° C. with 5% CO₂. Cells were washed with sterile 1×PBS and then resuspended in 100 μl culture media containing 1000 ng TCRmimics (RL4B, RL6A, RL1B), isotype controls (IgG2a, IgG1b) or an anti-HLA-A2 antibody (BB7.2a). The cells were incubated for 24 hrs at 37° C. with 5% CO₂. MTT (Promega, Madison, Wis.) was added to wells at a concentration of 10 μl in each well and allowed to develop overnight. Stop solution was added at 100 μl in each well, and plates were read for absorbance at 560 nm.

b) Cell Death assay. MDA-MB231 cells were plated on a 12 well plate at a density of 20,000 cells/well in 2 ml of culture media and allowed to adhere for 24 hrs at 37° C. incubator with 5% CO₂. Plates were washed with sterile 1×PBS and resuspended in 2 ml of culture media containing 1000 ng, 200 ng and 1000 ng respectively of TCRm antibodies RL4B, RL6A, and RL1B; 1000 ng of isotype controls (IgG2a, IgG1); 1000 ng of the anti-HLA-A2 antibody (BB7.2A); or were left untreated. Plates were allowed to incubate for 24 hrs, after which the media from each well was collected separately into 15 ml conical tubes. The cells were washed with 1×PBS followed by addition of trypsin to detach the cells. Media was added to neutralize the trypsin, and cells were centrifuged at 500 RPM for 5 minutes followed by removal of the supernatant and resuspension in 500 μl of 1× binding buffer. Annexin V-APC (BD Pharmingen) was added to each tube at an amount of 1.25 μl except for blank controls (untreated-unstained cells and untreated-PI stained cells), followed by incubation for 15 minutes in the dark. The cells were then washed resuspended in 500 μl of 1× binding buffer, followed by addition of 10 μl Propidium Iodide (PI) (BD Pharmingen) to each tube, except for blank controls (untreated-unstained cells and untreated-Annexin V stained cells). The samples were then immediately read on a FACS Canto followed by analysis on DIVA software (BD Biosciences).

c) Complement dependent cytotoxicity (CDC). CDC analysis of MDA-MB-231 cells using antibody dilutions and tetramer competition was conducted on adherent cells. Cells were plated and allowed to adhere overnight before Ab or Ab plus tetramer was applied. Antibody concentration was varied from 10 to 1.25 μg/ml, and tetramer concentration was kept constant at 6 μg/ml. CDC of cells incubated with Ab in the absence of tetramer showed an Ab concentration-dependent lysis which was paralleled by cells incubated with Ab in the presence of VLQ tetramer. Specific lysis in the CDC assays was calculated as follows: ([experimental release−spontaneous release]/[maximum release−spontaneous release])×100=specific release.

d) Antibody dependent cell-mediated cytotoxicity (ADCC). To investigate the ability of the 3.2G1 TCRm mAb to mediate ADCC, peripheral blood mononuclear cells (PBMCs) were isolated from the platelet chambers of apheresis collection devices from anonymous donors. The cells were held in serum-free medium (AIM-V) containing 200 U/ml recombinant human IL-2 for 2-7 days, with medium changes every 2-3 days to maintain and activate the NK population. ADCC reactions using human PBMC effector cells were conducted on MDA-MB-231 target cells using TCRm (RL4B) and positive control (W6/32) at a final concentration of 10 μg/ml. E:T ratios were 20:1. Specific lysis was calculated using ([E+T+Ab release−E+T−Ab release]/[maximum release−spontaneous release])×100=specific release. Final results were normalized to IgG2a isotype control by subtraction of background.

In vivo studies.

a) Prevention. Athymic nude mice (CByJ.Cg-Foxn1 {V}/j) were obtained from The Jackson Laboratory and housed under sterile conditions in barrier cages. Nineteen mice were implanted with 5×10⁶ freshly harvested MDA-MB-231 cells at 97% viability in Matrigel (Sigma-Aldrich). Mice received an i.p. injection of either 100 μg of an isotype IgG2a control Ab (n=10) or 100 μg of 3.2G1 (n=9) at the same time the tumor was implanted in the neck s.c. and 50 μg of either 3.2G1 or isotype control Ab weekly for the following 3 wk (total injections=4). Animals were held for at least 1 week after the appearance of the last tumor in the isotype control group (a total of 70 days) before totaling frequency of occurrence. All tumors reached ≧3 mm in diameter before being scored as positive.

b) Treatment. Athymic nude mice (CByJ.Cg-Foxn1 {v}/j) were obtained from the Jackson Laboratory and housed under sterile conditions in barrier cages. Fifty mice were subcutaneously injected with 5×10⁶ freshly harvested MDA-MB-231 cells at 97% viability in Matrigel in the right mammary pads of mice. Mice received i.p. injection of either 100 μg of an isotype IgG2a control antibody (n=15) or 100 μg of RL4B (n=18), followed by 50 μg weekly for a total of 5 weeks. Injections were started after the tumors reached ≧35 mm³. Similarly, separate mice were injected i.p. with either 100 μg of isotype IgG2a control antibody (n=4) or 100 μg of RL6A (n=3).

c) Crossover/Debulking. The isotype group (n=5) treated with 100 μg of IgG2a followed by 50 μg weekly doses for a total of 5 weeks was then dosed weekly with 500 μg of RL6A at a concentration of 5 μg/μl in 1×PBS for another 5 weeks.

Statistical analysis. Results are expressed as the mean±S.D. Student's t-tests were used to determine significance among the groups. A value of p<0.05 was considered significant. Analyses were performed using GraphPad Prism software (GraphPad Software Inc, La Jollia, Calif.).

### Example 2

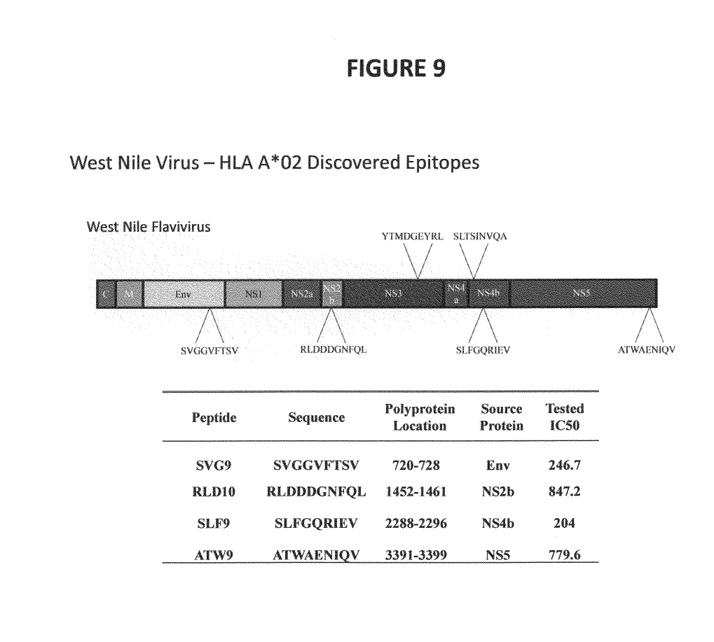

Characterization of TCRms against West Nile Virus epitopes. As depicted in FIG. 9, a total of four nonamer peptides having strong binding affinity for HLA-A*0201 were identified. Two peptides designated as WNV3 (SVGGVFTSV; SEQ ID NO:63) and WNV6 (SLFGQRIEV; SEQ ID NO:82) were used to generate TCRm antibodies. After screening hybridoma candidates RL14C (anti-WNV6 peptide/HLA-A2) and RL15A (anti-WNV3 peptide/HLA-A2) were identified and further characterized for binding specificity and affinity (see figures that follow).

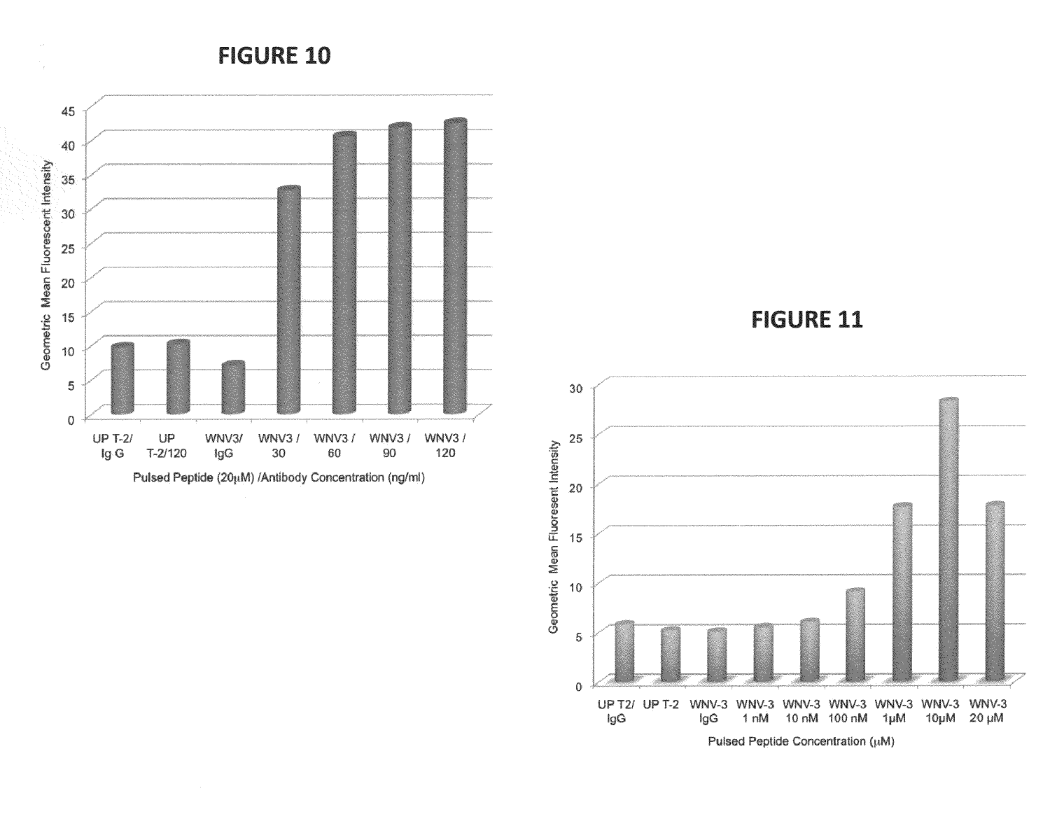

Purified RL15A was used at concentrations of 120, 90, 60 and 30 ng/ml to stain T2 cells pulsed with 20 μM of WNV-3 peptide, as shown in FIG. 10. RL15A TCRm showed optimum staining of WNV3 peptide pulsed T2 cells at a concentration of 120 ng/ml and fluorescence intensity decreased with titration of the TCRm concentration. Background staining was established using unpulsed T2 cells stained with either RL15A TCRm (120 ng/ml) or with mouse IgG₁ isotype control antibody (120 ng/ml). Data are representative of 3 independent experiments.

Next, the effect of peptide epitope density on TCRm staining intensity was examined, as depicted in FIG. 11. In this study, T2 cells were pulsed for four hours with WNV-3 peptide at concentrations ranging from 1 to 2×10⁴ nM and then stained with purified RL15A (120 ng/ml). Maximum RL15A TCRm staining was observed with T2 cells pulsed with 1×10⁴ nM of WNV3 peptide. Fluorescent signal was weakly detected for T2 cells pulsed with 10 nM of peptide. Background staining was determined using unpulsed T2 cells (UP T2) peptide and stained with RL15A TCRm (120 ng/ml) or with mouse IgG₁ isotype control antibody (120 ng/ml). In addition, no staining was detected using T2 cells pulsed with 2×10⁴ nM of WNV-3 peptide and stained with mouse isotope control antibody. Data are representative of 3 independent experiments.

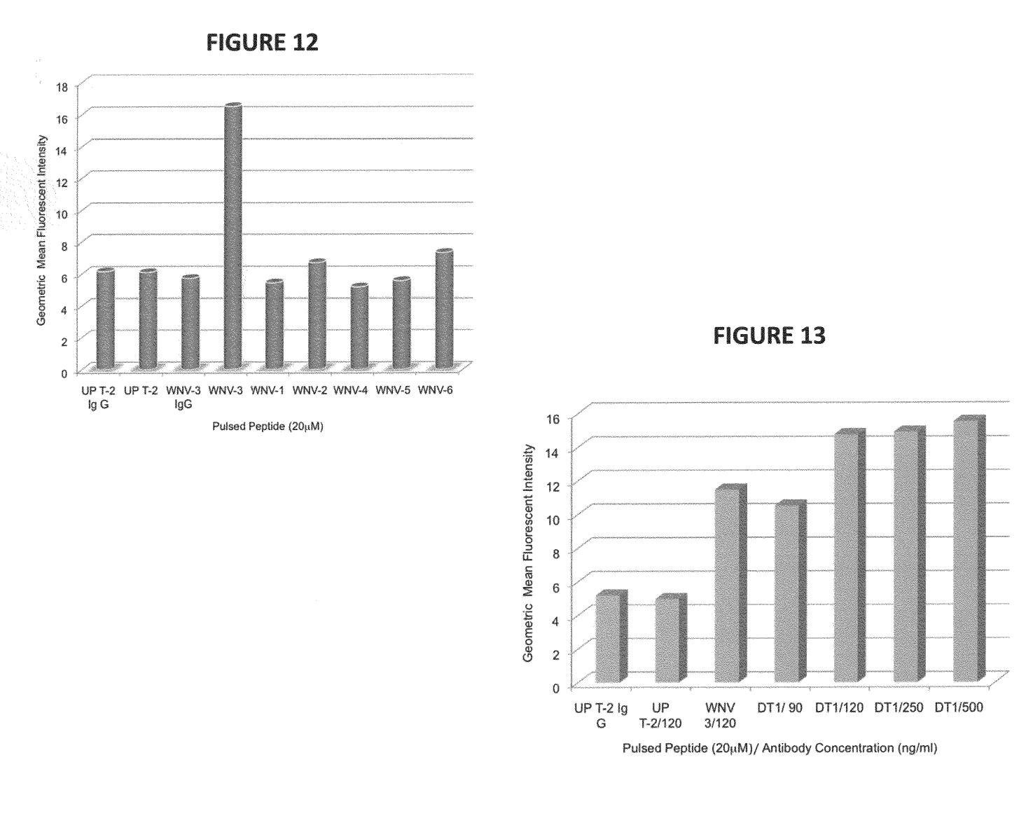

RL15A TCRm cross-reactivity for five other WNV peptides that bind HLA-A2 complexes with high affinity was then examined, as shown in FIG. 12. T2 cells were pulsed with WNV- peptides (1, 2, 3, 4, 5 & 6; SEQ ID NOS: 78, 79, 63, 80, 81 and 82, respectively) at 20 μM concentration and then stained with purified RL15A (120 ng/ml). RL15A stained only T2 cells pulsed with WNV-3 peptide (geometric mean fluorescent intensity fluorescent intensity˜16). RL15A TCRm did not stain T2 cells without peptide or pulsed with WNV peptides 1, 2, 4, 5, & 6. Background signal was determined using unpulsed T2 (UP T2) cells or pulsed with WNV-3 peptide (SEQ ID NO:63) and then stained with mouse isotype control antibody (120 ng/ml). Data are representative of 3 independent experiments.

Next, RL15A crossreactivity with other flavivirus peptides was investigated, as shown in FIGS. 13-19.

T2 cells were pulsed with dengue type-1 peptide (DT1; SEQ ID NO:57) at 20 μM concentration and then stained with purified RL15A at the following concentrations (90, 120, 250 and 500 ng/ml). As shown in FIG. 13, the maximal signal (geometric mean fluorescent intensity fluorescent intensity˜15) for DT1 peptide pulsed T2 cells was observed using 500 ng/ml of RL15A TCRm. Signal strength was greater for DT1 peptide (geometric mean fluorescent intensity fluorescent intensity>14) than WNV-3 peptide (geometric mean fluorescent intensity fluorescent intensity˜11) pulsed T2 cells when stained with RL15A TCRm at 120 ng/ml. Background signal was determined using unpulsed T2 (UP T2) cells stained with RL15A TCRm or mouse isotype control antibody (120 ng/ml). Data are representative of 3 independent experiments.

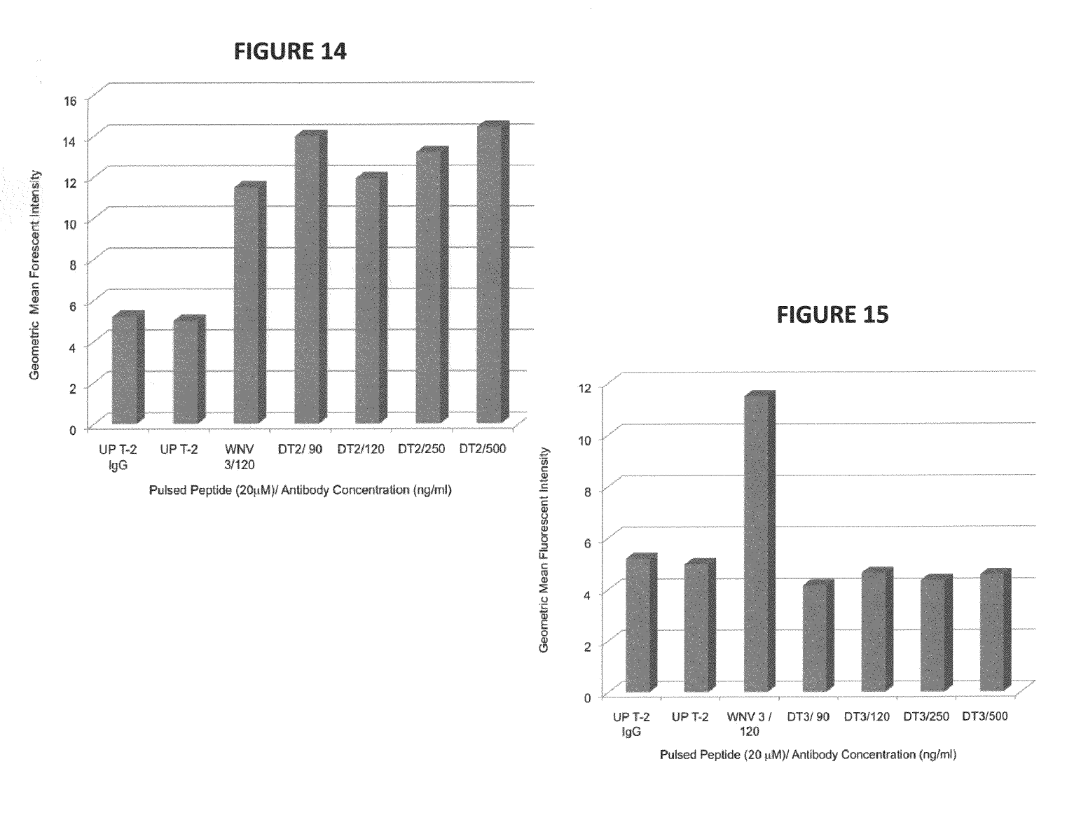

In FIG. 14, T2 cells were pulsed with dengue type-2 peptide (DT2; SEQ ID NO:89) at 20 μM concentration and then stained with purified RL15A at the following concentrations (90, 120, 250 and 500 ng/ml). Maximum signal (geometric mean fluorescent intensity˜14) for DT2 peptide pulsed T2 cells was observed using 500 ng/ml of RL15A TCRm. Signal strength was slightly greater for DT2 peptide (geometric mean fluorescent intensity˜11.5) than WNV-3 peptide (geometric mean fluorescent intensity˜11) pulsed T2 cells when stained with RL15A TCRm at 120 ng/ml. Background signal was established using unpulsed T2 (UP T2) cells stained with RL15A TCRm or mouse isotype control antibody (120 ng/ml). Data are representative of 3 independent experiments.

In FIG. 15, T2 cells were pulsed with dengue type-3 peptide (DT3; SEQ ID NO:90) at 20 μM concentration and then stained with purified RL15A at the following concentrations (90, 120, 250 and 500 ng/ml). RL15A binding to T2 cells pulsed with DT3 was not observed as the strength of signal was equal to or less than background signal determined using unpulsed T2 (UP T2) cells stained with RL15A TCRm or mouse isotype control antibody (120 ng/ml). Data are representative of 3 independent experiments.

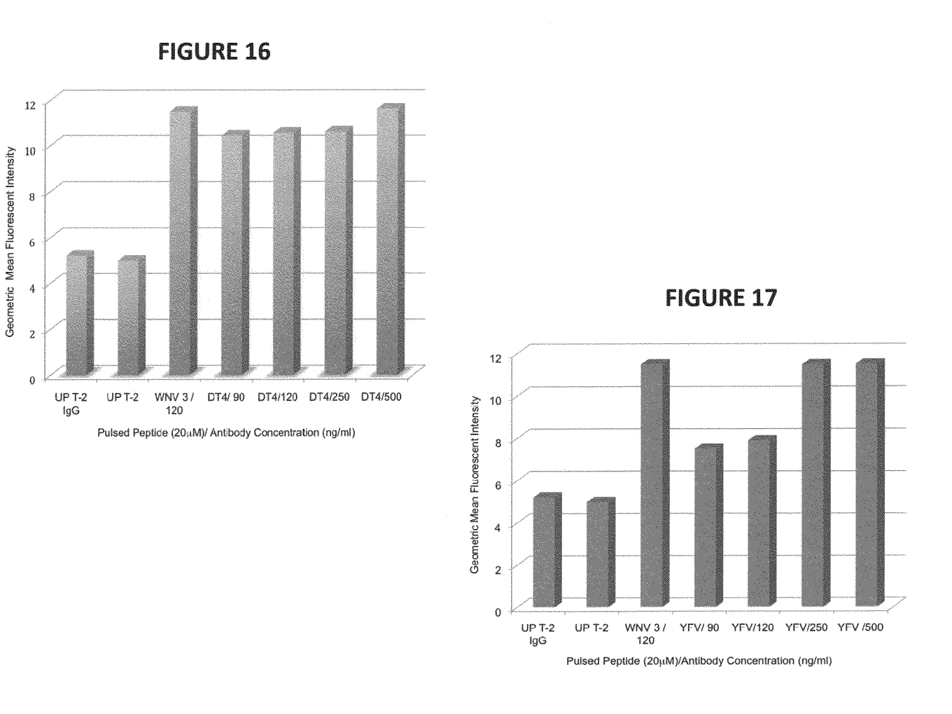

In FIG. 16, T2 cells were pulsed with dengue type-4 peptide (DT4; SEQ ID NO:91) at 20 μM concentration and then stained with purified RL15A at the following concentrations (90, 120, 250 and 500 ng/ml). Maximum signal (geometric mean fluorescent intensity˜11) for DT4 peptide pulsed T2 cells was observed using 500 ng/ml of RL15A TCRm. Little if any signal variation was seen with respect to staining DT4 peptide pulsed T2 cells with various concentrations of RL15A. Background signal was established using unpulsed T2 cells (UP T2) stained with RL15A TCRm or mouse isotype control antibody (120 ng/ml). Data are representative of 3 independent experiments

In FIG. 17, T2 cells were pulsed with yellow fever virus peptide (YFV; SEQ ID NO:94) at 20 μM concentration and then stained with purified RL15A at the following concentrations (90, 120, 250 and 500 ng/ml). Maximum signal (geometric mean fluorescent intensity˜11) for YFV peptide pulsed T2 cells was observed using 250 & 500 ng/ml of RL15A TCRm. Background signal was established using unpulsed T2 (UP T2) cells stained with RL15A TCRm or mouse isotype control antibody (120 ng/ml). Detection of RL15A TCRm and mouse isotype control antibody binding was detected using goat-anti-mouse IgG-PE conjugate (500 ng/ml) and the geometric mean fluorescent intensities determined by flow cytometric analysis were plotted. Data are representative of 3 independent experiments.

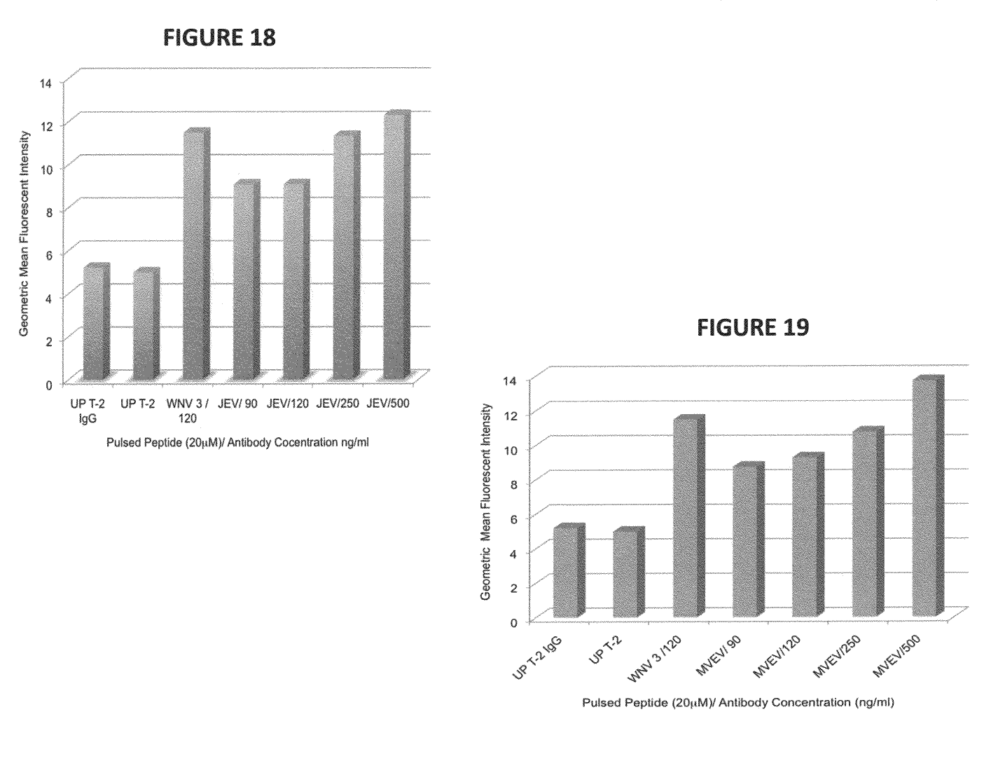

In FIG. 18, T2 cells were pulsed with JEV/SEV peptide (SEQ ID NO:92) at 20 μM concentration and then stained with purified RL15A at the following concentrations (90, 120, 250 and 500 ng/ml). Maximum signal (geometric mean fluorescent intensity˜12) for JEV/SEV peptide pulsed T2 cells was observed using 500 ng/ml of RL15A TCRm. Background signal was established using unpulsed T2 (UP T2) cells stained with RL15A TCRm or mouse isotype control antibody (120 ng/ml). Data are representative of 3 independent experiments.

In FIG. 19, T2 cells were pulsed with Murray Valley Encephalitis virus (MVEV; SEQ ID NO:93) at 20 μM concentration and then stained with purified RL15A at the following concentrations (90, 120, 250 and 500 ng/ml). Maximum signal (geometric mean fluorescent intensity˜13) for MVEV peptide pulsed T2 cells was observed using 500 ng/ml of RL15A TCRm. Background signal was established using unpulsed T2 (UP T2) cells stained with RL15A TCRm or mouse isotype control antibody (120 ng/ml). Data are representative of 3 independent experiments.

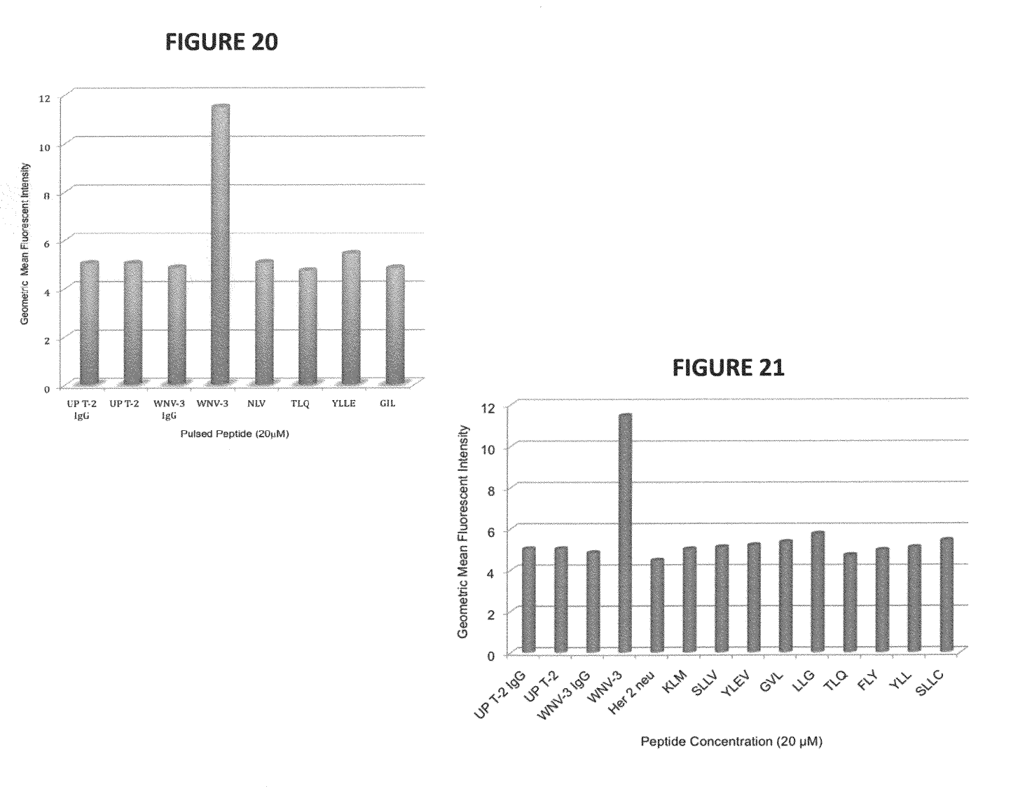

Next, RL5A crossreactivity to non-related viral peptides was investigated. In FIG. 20, T2 cells were pulsed with 20 μM of the following peptides: WNV-3 (SEQ ID NO:63), CMVpp65 (NLVPMWATV; SEQ ID NO:95), HPV18 E7-1 (TLQDIVLHL; SEQ ID NO:66), Epstein Barr Virus (YLLEMLWRL; SEQ ID NO:76) and Influenza M1 (GILGFVRL; SEQ ID NO:28) and stained with purified RL15A (120 ng/ml). RL15A TCRm cross-reactivity to non-related viral peptides was not observed as geometric mean fluorescent intensity values were comparable to background single intensity. Background signal was determined by staining unpulsed T2 (UP T2) cells with RL15A TCRm (120 ng/ml) or with mouse IgG₁ isotype control antibody (120 ng/ml). In addition, only background signal was detected for T2 cells pulsed with 20 μM of WNV-3 peptide and stained with mouse isotype control antibody. Data are representative of 3 independent experiments.

In FIG. 21, T2 cells were pulsed with 20 μM of the following peptides: WNV-3 (SEQ ID NO:63), Her2/neu (KIFGSLAFL; SEQ ID NO:5), CD19 (KLMSPKLYV; SEQ ID NO:46), NY-ESO-1 (SLLMWIQTV; SEQ ID NO:96), gP100 (YLEVGPVTA; SEQ ID NO:97), human chorionic gonadotropin-β (GVLPALPQV; SEQ ID NO:4), p53 tumor suppressor protein (LLGRNSFEV; SEQ ID NO:8), human chorionic gonadotropin-β (VLQGVLPAL; SEQ ID NO:3), topoisomerase (FLYDDNQRV; SEQ ID NO:27), p68 (YLLPAIVHI; SEQ ID NO:10), NY-ESO-1 wild-type (SLLMWIQTC; SEQ ID NO:59) and stained with purified RL15A (120 ng/ml). RL15A TCRm cross-reactivity to non-related viral peptides was not observed as geometric mean fluorescent intensity values were comparable to background single intensity. Background signal was determined by staining unpulsed T2 (UP T2) with RL15A TCRm (120 ng/ml) or with mouse IgG₁ isotype control antibody (120 ng/ml). In addition, only background signal was detected for T2 cells pulsed with 20 μM of WNV-3 peptide and stained with mouse isotype control antibody. Data are representative of 3 independent experiments.

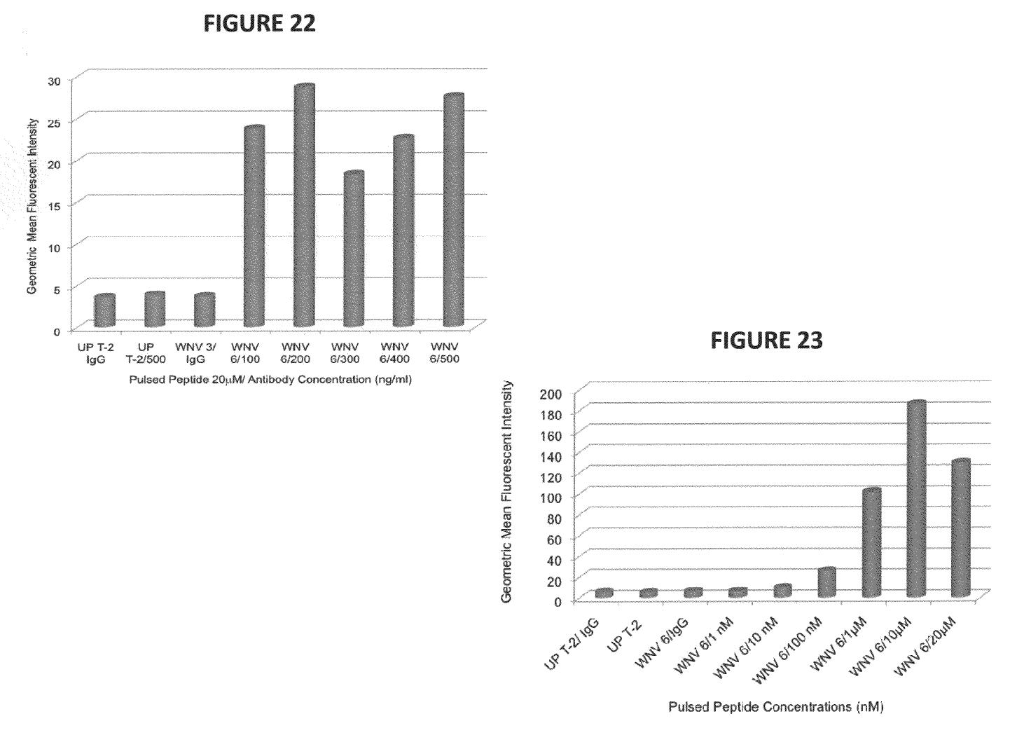

In the next set of Figures, monoclonal antibody RL14C was further characterized. In FIG. 22, purified RL14C was used at concentrations of 100, 200, 300, 400, and 500 ng/ml to stain T2 cells pulsed with 20 μM of WNV-6 peptide (SEQ ID NO:82). Background signal was established using unpulsed T2 (UP T2) cells stained with either RL14C TCRm (500 ng/ml) or with mouse IgG₁ isotype control antibody (500 ng/ml). Data are representative of 3 independent experiments.

In FIG. 23, T2 cells were pulsed for four hours with WNV-6 peptide (SEQ ID NO:82) at concentrations ranging from 1 to 2×10⁴ nM and then stained with purified RL14C (200 ng/ml). Staining signal was weak for T2 cells pulsed with 10 nM of WNV6 peptide. Background signal was determined using unpulsed T2 (UP T2) cells and stained with RL14C TCRm (200 ng/ml) or with mouse IgG₁ isotype control antibody (200 ng/ml). In addition, no signal was detected using T2 cells pulsed with 2×10⁴ nM of WNV-6 peptide and stained with mouse isotope control antibody. Data are representative of 3 independent experiments.

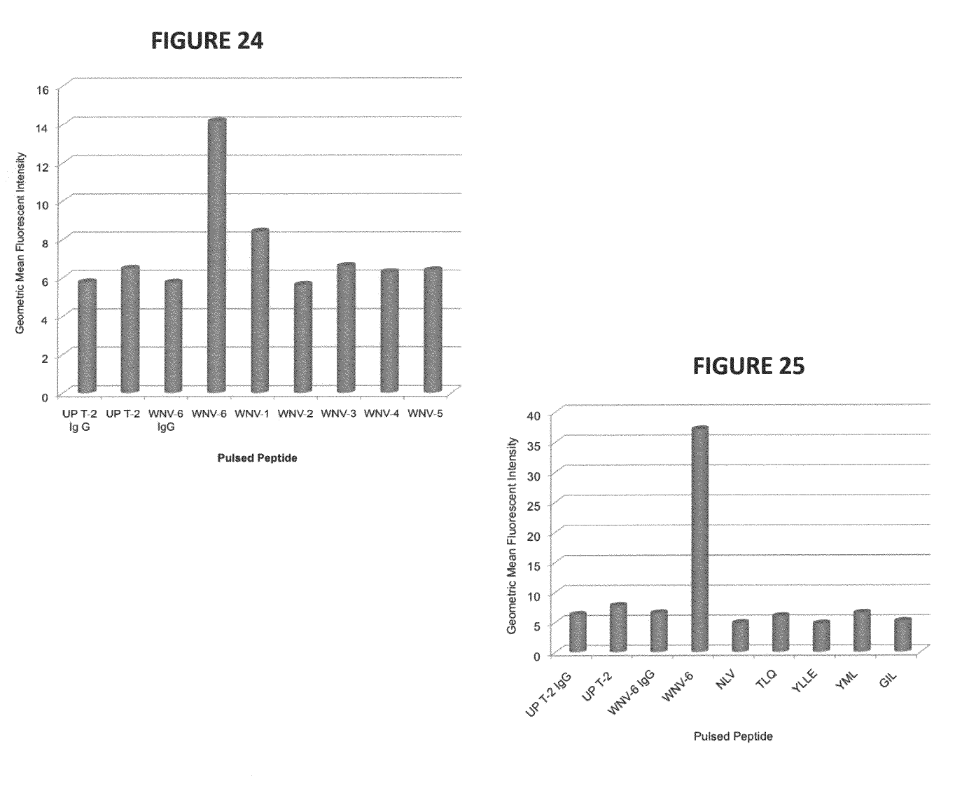

RL14C TCRm crossreactivity for five other WNV peptides that bind HLA-A2 complex with high affinity was examined. In FIG. 24, T2 cells were pulsed with WNV-peptides (1, 2, 3, 4, 5, & 6; SEQ ID NOS:78, 79, 63, 80, 81, and 82, respectively) at 20 μM concentration and then stained with purified RL14C (200 ng/ml). RL14C stained only T2 cells pulsed with WNV-6 peptide (geometric mean fluorescent intensity˜14). RL14C TCRm did not stain T2 cells without peptide or pulsed with WNV peptides 1, 2, 3, 4 & 5. Background signal was determined using unpulsed T2 (UP T2) cells or pulsed with WNV-6 peptide and then stained with mouse isotype control antibody (200 ng/ml). Data are representative of 3 independent experiments.

Next, RL14C TCRm crossreactivity with non-related viral peptide was investigated. In FIG. 25, T2 cells were pulsed with 20 mM of the following peptides: WNV-6 (SEQ ID NO:82), CMVpp65 (NLVPMWATV; SEQ ID NO:95), HPV18 E7-1 (TLQDIVLHL; SEQ ID NO:66), Epstein Barr Virus (YLLEMLWRL; SEQ ID NO:76), HPV16 (YMLDLQPETT; SEQ ID NO:77) and Influenza M1 (GILGFVTL; SEQ ID NO:28) and stained with purified RL14C (200 ng/ml). RL14C TCRm cross-reactivity to non-related viral peptides was not observed as geometric mean fluorescent intensity values were comparable to background signal intensity. Background signal was determined by staining unpulsed T2 (UP T2) cells with RL14C TCRm (200 ng/ml) or with mouse IgG₁ isotype control antibody (200 ng/ml). In addition, only background signal was detected for T2 cells pulsed with 20 μM of WNV-6 peptide and stained with mouse isotype control antibody. Data are representative of 3 independent experiments.

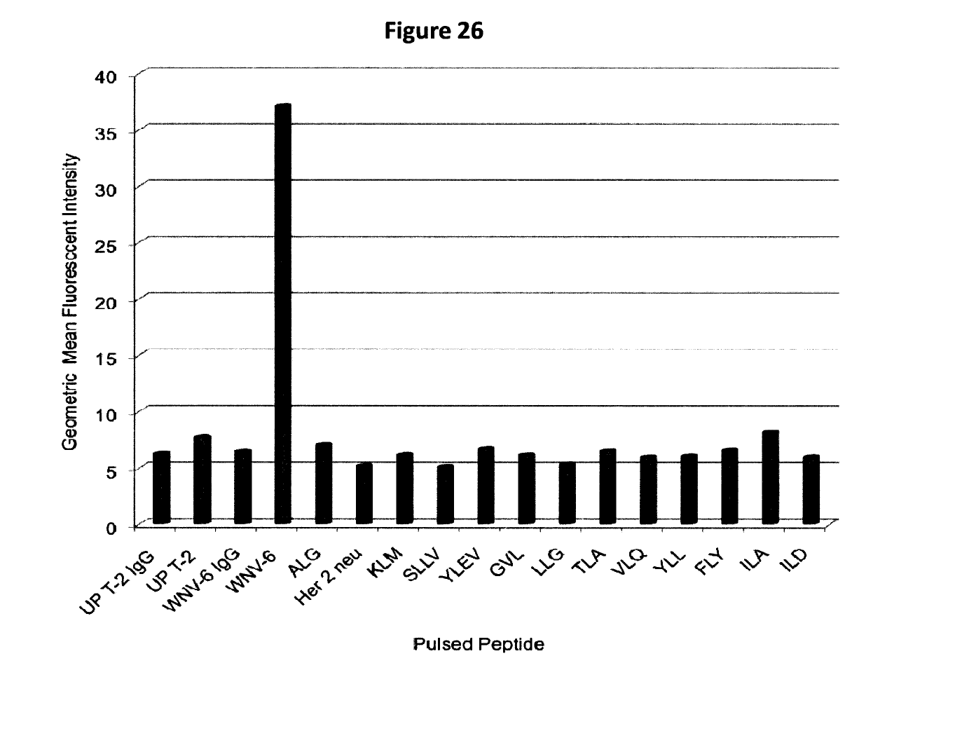

In FIG. 26, T2 cells were pulsed with 20 μM of the following peptides: WNV-6, MART-1 (ALGIGILTV; SEQ ID NO:17), Her2/neu (KIFGSLAFL; SEQ ID NO:5), CD19 (KLMSPKLYV; SEQ ID NO:46), NY-ESO-1 (SLLMWIQTV; SEQ ID NO:59), gP100 (YLEVGPVTA; SEQ ID NO:97), human chorionic gonadotropin-β (GVLPALPQV; SEQ ID NO:4), p53 tumor suppressor protein (LLGRNSFEV; SEQ ID NO:8), CD19 (TLAYLIFCL; SEQ ID NO:11), human chorionic gonadotropin-β (VLQGVLPAL; SEQ ID NO:3), p68 (YLLPAIVHI; SEQ ID NO:10), topoisomerase (FLYDDNQRV; SEQ ID NO:27), hTERT:540 (ILAKFLHWL; SEQ ID NO:14), and ODC-1 (ILDQKINEV; SEQ ID NO:33) and stained with purified RL14C (200 ng/ml). RL14C TCRm cross-reactivity to non-related cancer-associated peptides was not observed, as geometric mean fluorescent intensity values were comparable to background signal intensity. Background signal was determined by staining unpulsed T2 (UP T2) cells with RL14C TCRm (200 ng/ml) or with mouse IgG₁ isotype control antibody (200 ng/ml). In addition, only background signal was detected for T2 cells pulsed with 20 μM of WNV-6 peptide and stained with mouse isotype control antibody. Data are representative of 3 independent experiments.

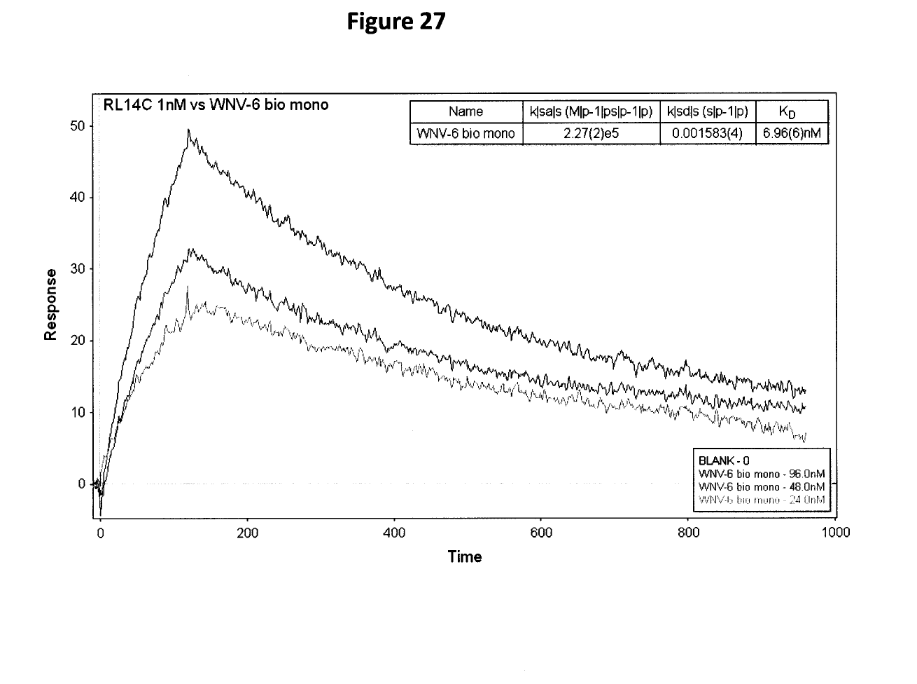

The affinity for RL14C was determined using surface plasmon resonance, as shown in FIG. 27. In brief, a rat anti-mouse IgG antibody was immobilized to a sensor chip via standard amine coupling chemistry. A 10 nM solution of RL14C TCRm was passed over the sensor chip and captured by the immobilized anti-mouse IgG mAb. Then WNV-6 peptide/HLA-A2 monomer complexes at 24, 48 and 96 nM were run over the chip, and binding to RL14C was observed and reported as response units (RU). The rates of association and dissociation were determined as 2.27×10⁵ and 1.58×10⁻³, respectively. The affinity constant K_{D}=6.96 nM and the t_{1/2} rate of dissociation=438 seconds.

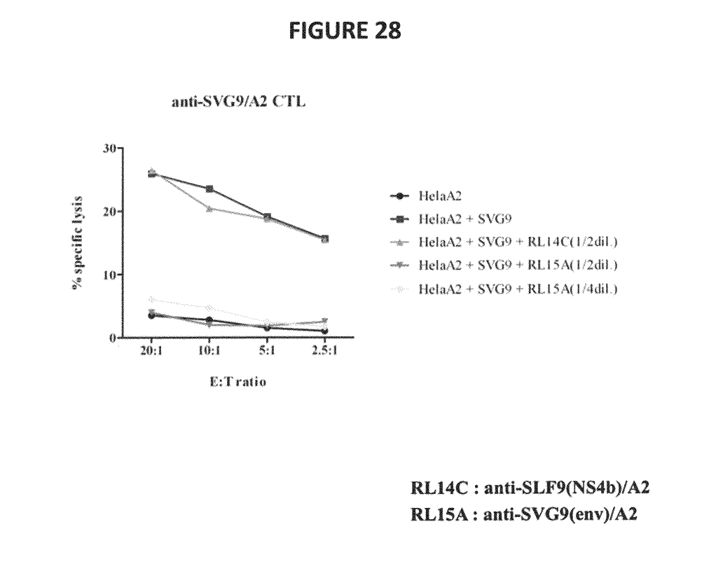

The fine binding specificity of RL15A TCRm has been demonstrated previously herein. In FIG. 28, these observations were expounded upon by demonstrating the ability of the TCRm to inhibit in a specific manner the stimulation of a WNV3 peptide/HLA-A2 reactive CTL cell line. HelaA2 cells were pulsed with WNV3 peptide or not pulsed with peptide and then incubated with the CTL cell line at effector to target ratios ranging from 20:1 to 2.5:1. CTL incubated with WNV3 peptide-pulsed HelaA2 cells lysed target cells in a dose dependent manner. When RL15A TCRm was included in the incubation mix, the TCRm blocked CTL killing of target cells. In contrast, RL14C TCRm specific for WNV6 peptide/HLA-A2 did not inhibit CTL lyses of target cells. These data strongly support the fine recognition specificity of the RL15A TCRm.

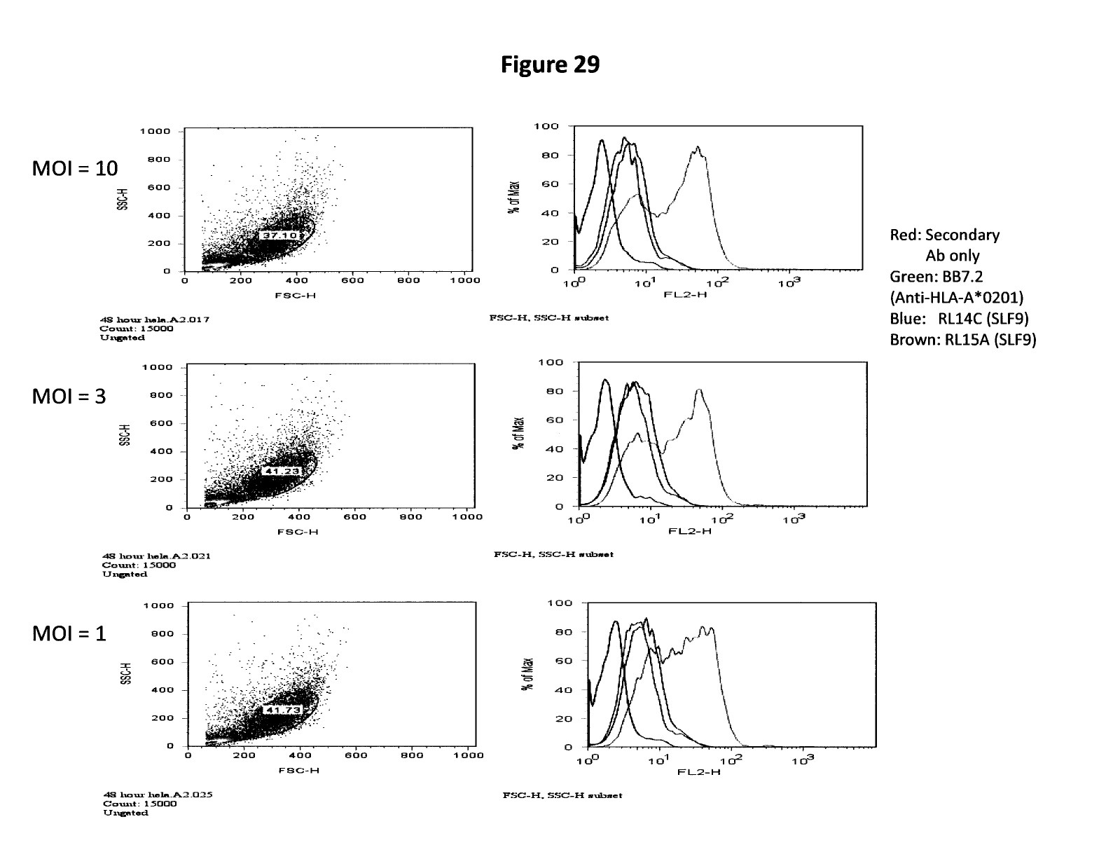

While critical data on the RL15A TCRm binding specificity has been presented, the data still have not demonstrated that 1) the WNV3 and WNV6 peptide epitopes are displayed on WNV infected cells and 2) the RL14C and RL15A TCRms can detect the naturally processed and presented WNV epitopes on infected cells. In this experiment, HelaA2 cells were infected with WNV at MOIs of 10, 3 and 1 and then stained with RL14C and RL15A at 48 h post-infection. Detection of bound TCRm was carried out using a goat-anti-murine IgG1-PE conjugate and flow cytometric analysis. The data presented in FIG. 29 reveal for the first time the presentation of two novel WNV peptide epitopes on infected cells using TCRms. Cells were also stained with the pan-HLA-A2 antibody, BB7.2, which showed maximum HLA-A2 expression. As expected, the TCRm staining profiles were markedly weaker than that of the BB7.2 mAb. In any event, the TCRm detected both WNV3 and WNV6 peptide epitopes on viral infected cells.

### Materials and Methods for Example 2

Viral-infected cell staining for flow cytometry. Purified TCRms RL15A or RL14C were used at concentrations ranging from 30-120 ng/ml to stain T2 cells pulsed with various concentrations of selected WNV peptides, selected flavivirus peptides, cancer-associated peptides, or irrelevant peptides, as indicated by the figures. Background staining was established using unpulsed T2 cells (UP T2) stained with either TCRm of interest (120 ng/ml or as indicated in figure) or with mouse IgG₁ isotype control antibody (120 ng/ml or as indicated in figure). TCRm binding was detected using goat-anti-mouse IgG-PE conjugate (250-500 ng/ml), and the geometric mean fluorescent intensity (GMFI) was determined by flow cytometric analysis utilizing either a FACS Canto or FACS Scan (BD Biosciences). Data were analyzed by either FACS Diva or CellQuest software (BD Biosciences) and are representative of 3 independent experiments. For natural infections of WNV, HelaA2 cells were infected with WNV at MOIs of 10, 3 and 1 and then stained with RL14C and RL15A at 48 h post-infection. Detection of bound TCRm was carried out using a goat-anti-murine IgG1-PE conjugate and flow cytometric analysis.

Affinity determination using surface plasmon resonance. Rat anti-mouse IgG antibody was immobilized to a sensor chip via standard amine coupling chemistry. A 10 nM solution of RL14C TCRm was passed over the sensor chip and captured by the immobilized anti-mouse IgG mAb. Then WNV-6 peptide/HLA-A2 monomer complexes at 24, 48 and 96nM were run over the chip, and binding to RL14C was observed and reported as response units (RU), utilizing a SensiQ (ICx Nomadics, OKC). Association, disassociation, and affinity constants (K_{D}) were determined using manufacturer supplied analysis software and algorithms.

### Example 3

Immune modulation by TCRm represents a novel application for these agents that could be applied to the inhibition of antigen specific T cell responses. TCRm could be used to specifically block activation and expansion of auto-reactive T cells or T cells that are involved in mediating chronic inflammation. The use of TCRm in this manner represents a unique paradigm shifting technology and an approach to immunotherapy not previously conceived. Presented in the next section are several examples of TCRm specific for two different peptides derived from human chorionic gonadotropin beta (hCGβ) protein that show specific inhibition of antigen reactive CD8⁺ T cell lines.

Generation of TCRms, characterization of binding to specific peptide, and demonstration of target display on tumor cells. Following the synthesis of HLA-A2 tetramers loaded with peptide (TMT or GVL; SEQ ID NOS:2 or 4, respectively), splenocytes isolated from immunized mice were prepared for fusion with the P3X-63Ag8.653 myeloma cell line and plated in a semi-soft cellulose medium. After about two weeks, colonies were identified, picked to individual wells of a 96 well plate for expansion and the hybridoma supernatants were screened for reactive antibodies. Table II shows the results from hybridoma fusions for each peptide-HLA-A2 immunogen. Several IgG1, IgG2a and IgG2b antibodies were selected from each immunization group.

To determine the peptide-specific reactivity of RL3A (anti-TMT-A2) and RL4D (anti-GVL-A2), the mAbs were first purified by affinity chromatography on a protein-G column and their binding specificity assessed by ELISA. Each antibody (tested at 1 μg/ml) showed significant reactivity for its respective peptide without any detection of binding to the irrelevant peptides (data not shown). These findings demonstrate that each of the antibodies selected has no detectable cross reactivity with either the HLA complex or any of a series of HLA complexes loaded with various peptides, which also bind HLA-A2.

Although each TCRm recognizes its cognate peptide-A2 target in coated wells, it was unclear whether these mAbs would recognize the specific peptide when loaded into HLA-A*0201 complexes expressed on a cell surface. In order to ensure that these TCRms recognize their specific peptide in the context of the native HLA-A2, their binding to T2 cells pulsed with 20 μM of specific, irrelevant peptides or no peptide was analyzed. Both TCRms stain T2 cells pulsed with only specific peptide (data not shown). These results confirm the fine and unique specificity of each TCRm for their respective peptide present in the HLA-A2 complex.

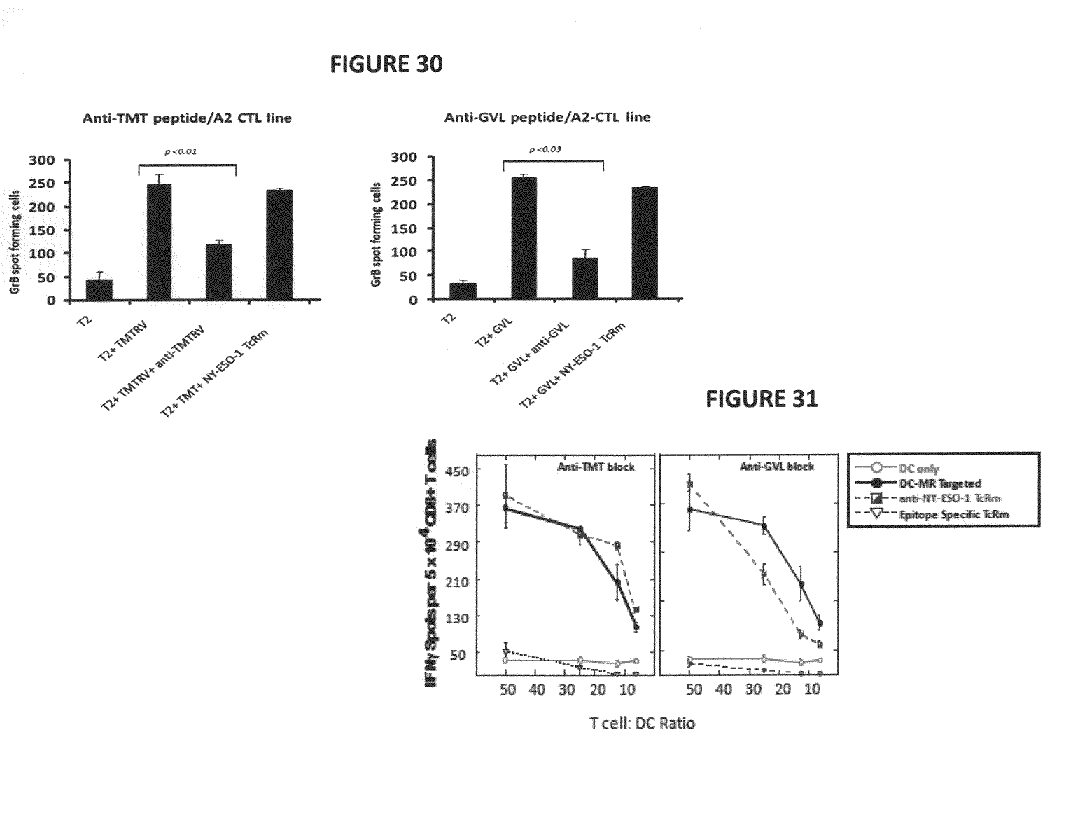

Inhibition of CTL stimulation with peptide-epitope specific TCRm. CTL lines were generated against the TMT and GVL peptide-HLA-A*0201 epitopes using autologous dendritic cells. CTL peptide specificity was determined using T2 cells alone or T2 cells pulsed with relevant peptide. As shown in FIG. 30, TMT and GVL peptide-specific CTL lines responded to T2 cells presenting relevant peptide but not to T2 cells alone. Further, granzyme B production by CTL lines specific for TMT and GVL peptide-epitopes was inhibited by the addition of anti-TMT and anti-GVL TCRm, respectively. In this study peptide-epitope specific TCRm were used to confirm CTL recognition specificity for the TMT peptide and GVL peptide epitopes.

Peptide-specific CTL recognize TMT and GVL peptide-HLA-A2 complexes on vaccine treated autologous DCs. To this point it had been demonstrated that vaccine targeted DC could stimulate anti-hCGβ CTL, demonstrating that the DCs were processing and presenting peptides from the hCGβ vaccine construct. To determine whether the TMT and/or GVL peptides were endogenously processed and presented, autologous DCs were treated with the B11-hCGβ vaccine conjugate, and CTL were assessed for IFN-γ production. As shown in FIG. 31, the CTL response was specific for TMT peptide and GVL peptide epitopes and directly correlated with effector cell to target cell ratio (E:T). Furthermore, the response was inhibited using the respected TMT or GVL peptide-epitope specific TCRm but not with control TCRm (anti-NY-ESO-1 (157-165; SEQ ID NO:59)-HLA-A2 TCRm). These findings indicate that TMT and GVL peptides are processed and presented in the context of HLA-A*0201 in vaccine-treated DCs and that TCRm antibodies are useful agents in validating the recognition specificity of the CTL response.

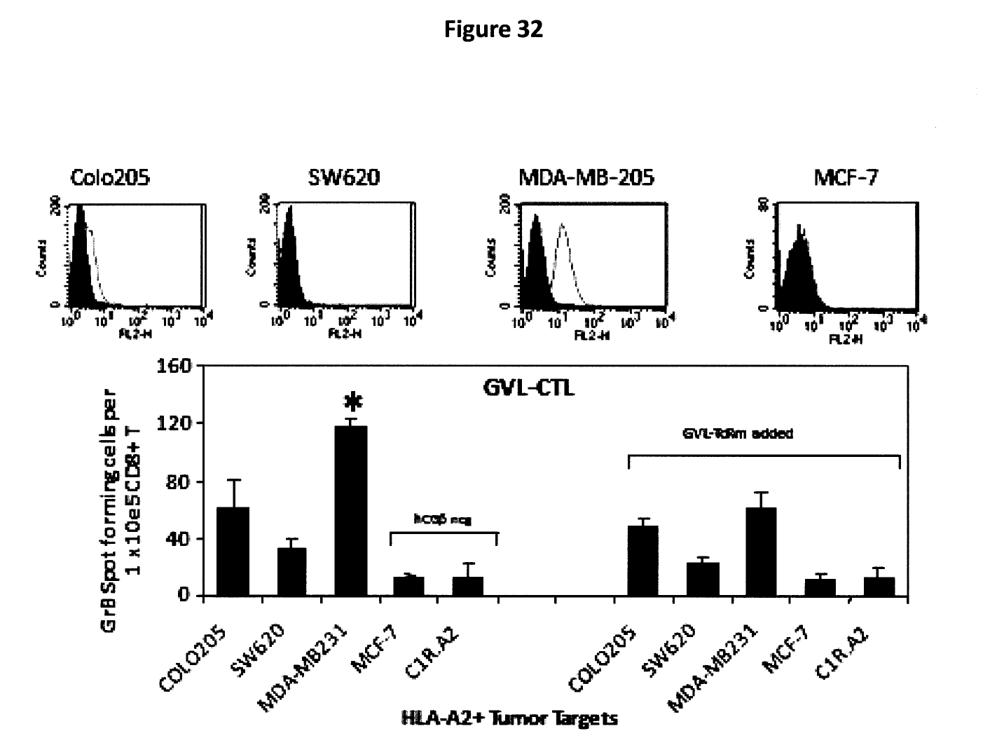

RL4D TCRm specifically blocks anti-GVL peptide/A2 CTL from reacting to tumor cell lines. Here, the ability of a TCRm to specially inhibit a CTL response to an antigen positive tumor cell line was examined. In the top panel of FIG. 32, the RL4D TCRm was used to stain the tumor cell lines. Only the Colo205 and MDA-MB-231 tumor cells showed staining with RL4D TCRm, demonstrating that the GVL peptide/A2 epitope was expressed on the surface of these cells (see histogram plots). The left side of the bottom panel shows GVL peptide-specific CTL production of granzyme B measured by ELISpot assay after 6 hr incubation with tumor cell lines. CTL incubated with MDA-MB-231 tumor cells produced granzyme B while none of the other tumor cell lines were able to stimulate CTL production of granzyme B enzyme. On the lower right hand panel, to verify that the CTL response was indeed GVL-peptide/HLA-A2 specific, cultures of CTL and tumor cells were incubated with RL4D TCRm (50 ng/ml). In this example the CTL response to the MDA-MB-231 cells was inhibited with the addition of the target specific TCRm.

### Materials and Methods for Example 3

Antibodies and synthetic peptides. The conjugated polyclonal antibodies goat anti-mouse-IgG (H+L chains)-horseradish peroxidase (HRP) and goat anti-mouse IgG heavy chain-phycoerythrin (PE) were purchased from Caltag Biosciences (Burlingame, Calif.). The mouse IgG₁ isotype control antibody was purchased from Southern Biotech (Birmingham, Ala.). Peptides TMTRVLQGV [residues 40-48, human chorionic gonadotropin-β peptide designated as TMT₍₄₀₎; (SEQ ID NO:2)] and GVLPALPQV [residues 47-55, human chorionic gonadotropin-β peptide, designated as GVL₍₄₇₎; (SEQ ID NO:4)] were synthesized at the University of Oklahoma Health Sciences Center, Oklahoma City, Okla., using a solid-phase method and purified by HPLC to greater than 90%.

Cell lines. The human lymphoblastoid cell line T2 (HLA-A*0201) and the P3X-63Ag8.653 murine myeloma cell line used as a fusion partner were purchased from the American Type Culture Collection (ATCC, Manassas, Va.).

Dendritic cells. Human peripheral blood mononuclear cells (PBMC) from anonymous donors were obtained from separation cones of discarded apheresis units from the Coffee Memorial Blood Center, Amarillo, Tex. after platelet harvest. Cells were separated on a ficoll gradient (Amersham Biosciences, Uppsala, Sweden), then washed, counted, typed for HLA-A2 by flow cytometry, and resuspended in AIM-V medium at 1-2×10⁷ cells/ml. PBMC were incubated in a T-80 (NalgeNunc, Rochester, N.Y.) or T-175 (Corning, Acton, Mass.) flask, depending on the volume, for 2 hours at 37° C. and 5% CO₂. Non-adherent cells were removed, the flask was washed twice with PBS, and then 15-30 ml supplemented AIM-V (10% heat-inactivated FBS, L-Glutamine and Pen/Strep) was added to the flask, as well as IL-4 (50 ng/ml) and GM-CSF (25 ng/ml), stimulating differentiation of monocytes into dendritic cells. Recombinant human IL-4 and GM-CSF were obtained from Peprotech (Rockyhill, N.J.). After 5-6 days, the immature dendritic cells were detached from the flask by incubation at 4° C. for 20-60 min., centrifuged, counted and either used immediately or frozen at −80° C. for later use.

Peptide specificity and sensitivity assays. T2 is a mutant cell line that lacks transporter-associated proteins (TAP) 1 and 2 which allows for efficient loading of exogenous peptides (Wei, M. L. & Cresswell, P. HLA-A2 molecules in an antigen-processing mutant cell contain signal sequence-derived peptides. Nature 1992, 356(6368), 443-446). The T2 cells were pulsed with the peptides at 20 μg/ml for 4 hours in growth medium, with the exception of the peptide-titration experiments, in which the peptide concentration was varied as indicated. Cells were washed and resuspended in staining buffer (SB; PBS+0.5% BSA+2 mM EDTA) and then stained with either a constant amount (1 μg) or a decreasing amount (4 mg -0.1 mg) of 3F9 or 1B10 TCRm antibody for 15 to 30 minutes in 100 μl SB. Cells were then washed with 3 ml SB, and the pellet was resuspended in 100 μl of SB containing 2 μl of either of two goat anti-mouse secondary antibodies (FITC or PE labeled). After incubating for 15-30 minutes at room temperature, the wash was repeated and cells were resuspended in 0.5 ml SB, analyzed on a FACScan instrument and evaluated using Cell Quest Software (BD Biosciences, Franklin Lakes, N.J.). To evaluate the peptide binding sensitivity of each TCRm, T2 cells were pulsed for 4 hours with decreasing amounts of specific peptide (2,000-0.15 nM). T2 cells (5×10⁵) were then washed in SB to remove excess peptide and stained with each TCRm-PE conjugate, 3F9 and 1B10 TCRms at 1 mg/mL of SB).

Analysis of Ag-specific T cells by IFN-γ and granzyme B ELISpot assay. T cells were stimulated as bulk cultures in vitro on a 8-10 day cycle for 3-4 weeks with autologous immature DCs previously exposed to the vaccine (contains hCGβ antigen) or the vehicle (no hCGβ antigen) at 30 μg/ml, two wells were untreated, and the plate was incubated for up to 3 days at 37° C., 5% CO₂ and matured with Poly I:C) at a ratio of 10:1 in the presence of cytokines sequentially added (10 ng/ml each of IL-7 on day 0 and IL-2 on day 1) every 3 days. Alternatively, CD8⁺ T cells from HLA-A2⁺ donors were repeatedly stimulated with hCGβ synthetic peptides (TMTRVLQGV (SEQ ID NO:2) and GVLPALPQV (SEQ ID NO:4)) loaded on to matured autologous DCs. Effector T lymphocytes were expanded on anti-CD3 and anti-CD28 Dynal immunomagnetic beads (Invitrogen, Carlsbad, Calif.) and restimulated with vaccine on day 14, and CD8⁺ and CD4⁺ T cells were purified using a commercial T cell enrichment kit (Miltenyi MACS, Auburn, Calif.). CTL activity of vaccine or peptide-stimulated CD8⁺ T cells was assessed using vaccine-treated DCs or peptide-loaded T2 cells in the presence of 3 μg/ml β2 microglobulin. CD8⁺ CTL response was measured in a cell-based IFN-γ cytokine or granzyme B production ELISpot assay (MabTech, Sweden and Cell Sciences, Canton, Mass. for ELISpot kits).

Statistical Analysis. The relationship between two parameters was tested using regression analysis, and a value of p<0.05 was considered significant. In the presence of a significant relationship, the coefficient of determination (R²) was calculated to express the degree of correlation.

Thus, in accordance with the presently disclosed and claimed invention, there has been provided a method of producing antibodies that recognize peptides associated with a tumorigenic or disease state, wherein the antibodies will mimic the specificity of a T cell receptor, that fully satisfies the objectives and advantages set forth hereinabove. Although the invention has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the invention.

### REFERENCES

The following references, to the extent that they provide exemplary procedural or other details supplementary to those set forth herein, are specifically incorporated herein by reference.
Adotevi O, Mollier K, Neuveut C, Cardinaud S, Boulanger E, Mignen B, et al. Immunogenic HLA-B*0702-restricted epitopes derived from human telomerase reverse transcriptase that elicit antitumor cytotoxic T-cell responses. Clin Cancer Res 2006; 12(10):3158-67.
Altman J D, Moss P A, Goulder P J, Barouch D H, McHeyzer-Williams M G, Bell J I, et al. Phenotypic analysis of antigen-specific T lymphocytes. Science 1996; 274(5284):94-6.
Andersen P S, Stryhn A, Hansen B E, Fugger L, Engberg J, Buus S. A recombinant antibody with the antigen-specific, major histocompatibility complex-restricted specificity of T cells. Proc Natl Acad Sci USA 1996; 93(5):1820-4.
Apostolopoulos, V., Haurum, J. S. & McKenzie, I. F. MUC1 peptide epitopes associated with five different H-2 class I molecules. Eur J Immunol, 27:2579-2587 (1997).
Bae, J., Martinson, J. A., and Klingemann, H. G. Identification of CD19 and CD20 peptides for induction of antigen-specific CTLs against B-cell malignancies. Clinical Cancer Research, 11(4):1629-38 (2005).
Barfoed, A. M., et al. Cytotoxic T-lymphocyte clones, established by stimulation with the HLA-A2 binding p5365-73 wild type peptide loaded on dendritic cells in vitro, specifically recognize and lyse HLA-A2 tumour cells overexpressing the p53 protein. Scand J Immunol, 51(2): 128-33 (2000).
Bates G J, Nicol S M, Wilson B J, Jacobs A M F, Bourdon J C, Lane D P, Perkins N D, Fuller-Pace F V. The DEAD box protein p68: a novel transcriptional coactivator of the p53 tumor suppressor. The EMBO Journal. 24: 543-553 (2005).
Biddison, W. E., et al. Tax and M1 peptide/HLA-A2-specific Fabs and T cell receptors recognize nonidentical structural features on peptide/HLA-A2 complexes. J Immunol, 171:3064-3074 (2003).
Bohm, C. M. et al. Identification of HLA-A2-restricted epitopes of the tumor-associated antigen MUC2 recognized by human cytotoxic T cells. Int J Cancer, 75:688-693 (1998).
Bonifaz L, Bonnyay D, Mahnke K, Rivera M, Nussenzweig M C, Steinman R M. Efficient targeting of protein antigen to the dendritic cell receptor DEC-205 in the steady state leads to antigen presentation on major histocompatibility complex class I products and peripheral CD8+ T cell tolerance. J Exp Med 2002; 196(12):1627-38.
Brooks, S. C., Locke, E. R. & Soule, H. D. Estrogen receptor in a human cell line (MCF-7) from breast carcinoma. J Biol Chem, 248:6251-6253 (1973).
Brossart, P. et al. Identification of HLA-A2-restricted T-cell epitopes derived from the MUC1 tumor antigen for broadly applicable vaccine therapies. Blood, 93:4309-4317 (1999).
Buchli R, Vangundy R S, Giberson C F, Hildebrand W H. Critical factors in the development of fluorescence polarization-based peptide binding assays: an equilibrium study monitoring specific peptide binding to soluble HLA-A*0201. J Immunol Methods 2006; 314(½):38-53.
Buchli R, VanGundy R S, Hickman-Miller H D, Giberson C F, Bardet W, Hildebrand W H. Development and validation of a fluorescence polarization-based competitive peptide-binding assay for HLA-A*0201—a new tool for epitope discovery. Biochemistry 2005; 44(37):12491-507.
Buchli R, VanGundy R S, Hickman-Miller H D, Giberson C F, Bardet W, Hildebrand W H. Real-time measurement of in vitro peptide binding to soluble HLA-A*0201 by fluorescence polarization. Biochemistry 2004; 43(46): 14852-63.
Bullock T N, Colella T A, Engelhard V H. The density of peptides displayed by dendritic cells affects immune responses to human tyrosinase and gp100 in HLA-A2 transgenic mice. J Immunol 2000; 164(5):2354-61.
Bullock T N, Mullins D W, Engelhard V H. Antigen density presented by dendritic cells in vivo differentially affects the number and avidity of primary, memory, and recall CD8+ T cells. J Immunol 2003;170(4):1822-9.
Bushell, M., et al. Cleavage of polypeptide chain initiation factor eIF4GI during apoptosis in lymphoma cells: characterization of an internal fragment generated by caspase-3-mediated cleavage.” Cell Death Differ, 7(7): 628-36 (2003).
Causevic M, Hislop R G, Kernohan N M, Carey F A, Kay R A, Steele R J C and Fuller-Pace F V. Overexpression and poly-ubiquitylation of the DEAD-box RNA helicase p68 in colorectal tumors. Oncogene 20: 7734-7743 (2001).
Celis E., V. Tsai, C. Crimi, R. DeMars, P. A. Wentworth, R. W. Chesnut, H. M. Grey, A. Sette, and H. M. Serra. “Induction of anti-tumor cytotoxic T lymphocytes in normal humans using primary cultures and synthetic peptide epitopes”. Proc. Natl. Acad. Sci. USA 1994; 91:2105-2109.
Chames, P. et al. TCR-like human antibodies expressed on human CTLs mediate antibody affinity-dependent cytolytic activity. J Immunol, 169:1110-1118 (2002).
Chikamatsu, K. et al. Generation of anti-p53 cytotoxic T lymphocytes from human peripheral blood using autologous dendritic cells. Clin Cancer Res, 5:1281-1288 (1999).
Chiriva-Internati M, Liu Y, Weidanz J A, Grizzi F, You H, Zhou W, et al. Testing recombinant adeno-associated virus-gene loading of dendritic cells for generating potent cytotoxic T lymphocytes against a prototype self-antigen, multiple myeloma HM1.24. Blood 2003; 102(9):3100-7.
Clark C E, Vonderheide R H. Getting to the surface: a link between tumor antigen discovery and natural presentation of peptide-MHC complexes. Clin Cancer Res 2005; 11(15):5333-6.
Clemens, M. J. Targets and mechanisms for the regulation of translation in malignant transformation.” Oncogene, 23(18): 3180-8 (2004).
Clinchy, B. et al. The growth and metastasis of human, HER-2/neu-overexpressing tumor cell lines in male SCID mice. Breast Cancer Res Treat, 61:217-228 (2000).
Cohen, C. J., Denkberg, G., Lev, A., Epel, M. & Reiter, Y. Recombinant antibodies with MHC-restricted, peptide-specific, T-cell receptor-like specificity: new tools to study antigen presentation and TCR-peptide-MHC interactions. J Mol Recognit, 16:324-332 (2003).
Coussens, L. et al. Tyrosine kinase receptor with extensive homology to EGF receptor shares chromosomal location with neu oncogene. Science, 230:1132-1139 (1985).
Cox et al. Identification of a peptide recognized by five melanoma-specific human cytotoxic T cell lines. Science, 264:716-719 (1994).
Dadaglio, G., et al., Characterization and quantitation of peptide-MHC complexes produced from hen egg lysozyme using a monoclonal antibody. Immunity, 6(6):727-38 (1997).
Dangles V, Halberstam I, Scardino A, Choppin J, Wertheimer M, Richon S, et al. Tumor-associated antigen human chorionic gonadotropin beta contains numerous antigenic determinants recognized by in vitro-induced CD8+ and CD4+ T lymphocytes. Cancer Immunol Immunother 2002; 50(12):673-81.
DeGroot et al. Rapid determination of HLA B*07 ligands from the West Nile virus NY99 genome. Emerging Infectious Diseases, 7:4 (2001).
DeLeo, A. B. p53-based immunotherapy of cancer. Crit Rev Immunol, 18:29-35 (1998).
Denkberg G, Cohen C J, Lev A, Chames P, Hoogenboom H R, Reiter Y. Direct visualization of distinct T cell epitopes derived from a melanoma tumor-associated antigen by using human recombinant antibodies with MHC-restricted T cell receptor like specificity. Proc Natl Acad Sci USA 2002; 99(14):9421-6.
Dhodapkar M V, Osman K, Teruya-Feldstein J, Filippa D, Hedvat C V, Iversen K, Kolb D, Geller M D, Hassoun H, Kewalramani T, Comenzo R L, Coplan K, Chen Y T, Jungbluth A A. Expression of cancer/testis (CT) antigens MAGE-A1, MAGE-A3, MAGE-A4, CT-7, and NY-ESO-1 in malignant gammopathies is heterogeneous and correlates with site, stage and risk status of disease. Cancer Immunity, 3:9-17 (2003).
Disis, M. L. et al. Existent T-cell and antibody immunity to HER-2/neu protein in patients with breast cancer. Cancer Res, 54:16-20 (1994).
Diwan, M. & Park, T. G. Stabilization of recombinant interferon-alpha by pegylation for encapsulation in PLGA microspheres. Int J Pharm, 252:111-122 (2003).
Dols, A. et al. Identification of tumor-specific antibodies in patients with breast cancer vaccinated with gene-modified allogeneic tumor cells. J Immunother, 26:163-170 (2003).
Dubsky P., H. Saito, M. Leogier, C. Dantin, J. E. Connolly, J. Banchereau, and A. K. Palucka. “IL-15-induced human DC efficiently prime melanoma-specific naïve CD8+ T cells to differentiate into CTL”. Eur. J. Immunol. 2007; 37:1678-1690.
Eastman, S., et al., A study of complexes of class II invariant chain peptide: major histocompatibility complex class II molecules using a new complex-specific monoclonal antibody. Eur J Immunol, 26(2):385-93 (1996).
Fingeroth, J. D. et al. Epstein-Barr virus receptor of human B lymphocytes is the C3d receptor CR2. Proc Natl Acad Sci USA, 81:4510-4514 (1984).
Fisk, B., Blevins, T. L., Wharton, J. T. & Ioannides, C. G. Identification of an immunodominant peptide of HER-2/neu protooncogene recognized by ovarian tumor-specific cytotoxic T lymphocyte lines. J Exp Med, 181:2109-2117 (1995).
Gauduin M C. Intracellular cytokine staining for the characterization and quantitation of antigen-specific T lymphocyte responses. Methods 2006; 38(4):263-73.
Glennie, M. J. & van de Winkel, J. G. Renaissance of cancer therapeutic antibodies. Drug Discov Today, 8:503-510 (2003).
Gnjatic, S. et al. Accumulation of the p53 protein allows recognition by human CTL of a wild-type p53 epitope presented by breast carcinomas and melanomas. J Immunol, 160:328-333 (1998).
Godelaine D, Carrasco J, Lucas S, Karanikas V, Schuler-Thurner B, Coulie P G, et al. Polyclonal CTL responses observed in melanoma patients vaccinated with dendritic cells pulsed with a MAGE-3.A1 peptide. J Immunol 2003; 171 (9):4893-7.
He L Z, Ramakrishna V, Connolly J E, Wang X T, Smith P A, Jones C L, et al. A novel human cancer vaccine elicits cellular responses to the tumor-associated antigen, human chorionic gonadotropin beta. Clin Cancer Res 2004; 10(6):1920-7.
Hersey P, Menzies S W, Coventry B, Nguyen T, Farrelly M, Collins S, et al. Phase I/II study of immunotherapy with T-cell peptide epitopes in patients with stage IV melanoma. Cancer Immunol Immunother 2005; 54(3):208-18.
Hickman, H. D. et al. C-terminal epitope tagging facilitates comparative ligand mapping from MHC class I positive cells. Hum Immunol, 61:1339-1346 (2000).
Hickman, H. D. et al. Cutting Edge: Class I presentation of host peptides following HIV infection. J Immunol, 171:22-26 (2003).
Hickman, H. D. et al. Toward a definition of self: proteomic evaluation of the class I peptide repertoire. J Immunol, 172:2944-2952 (2004).
Hinz T, Buchholz C J, van der Stappen T, Cichutek K, Kalinke U. Manufacturing and quality control of cell-based tumor vaccines: a scientific and a regulatory perspective. J Immunother 2006; 29(5):472-6.
Hoffmann, T. K., H. Bier, et al. p53 as an immunotherapeutic target in head and neck cancer. Adv Otorhinolaryngol, 62:151-60 (2005).
Irsch, J. et al. Isolation and characterization of allergen-binding cells from normal and allergic donors. Immunotechnology, 1:115-125 (1995).
Itoh K, Yamada A. Personalized peptide vaccines: a new therapeutic modality for cancer. Cancer Sci 2006; 97(10):970-6.
Jager, E., Chen, Y T, Drijfhout, J W, Karbach, J, Ringhoffer, M, Jager, D, Arand, M. et al. Simultaneous humoral and cellular immune response against cancer-testis antigen NY-ESO-1: definition of human histocompatibility leukocyte antigen (HLA)-A2-binding peptide epitopes. J Exp Med, 187:265-270 (1998).
Jager, E., Jager, D. & Knuth, A. Antigen-specific immunotherapy and cancer vaccines. Int J Cancer, 106:817-820 (2003).
Joseph, A. M., Babcock, G. J. & Thorley-Lawson, D. A. Cells expressing the Epstein-Barr virus growth program are present in and restricted to the naive B-cell subset of healthy tonsils. J Virol, 74:9964-9971 (2000).
Kageyama S, Tsomides T J, Sykulev Y, Eisen H N. Variations in the number of peptide-MHC class I complexes required to activate cytotoxic T cell responses. J Immunol 1995; 154(2):567-76.
Kearns-Jonker, M. et al. EBV binds to lymphocytes of transgenic mice that express the human CR2 gene. Virus Res, 50:85-94 (1997).
Keilholz U, Martus P, Scheibenbogen C. Immune monitoring of T-cell responses in cancer vaccine development. Clin Cancer Res 2006; 12(7 Part 2):2346s-52s.
Keilholz U, Weber J, Finke J H, Gabrilovich D I, Kast W M, Disis M L, et al. Immunologic monitoring of cancer vaccine therapy: results of a workshop sponsored by the Society for Biological Therapy. J Immunother 2002; 25(2): 97-138.
Knutson, K. L., Schiffman, K., Cheever, M. A. & Disis, M. L. Immunization of cancer patients with a HER-2/neu, HLA-A2 peptide, p369-377, results in short-lived peptide-specific immunity. Clin Cancer Res, 8:1014-1018 (2002).
Kodituwakku, A. P., Jessup, C., Zola, H. & Roberton, D. M. Isolation of antigen-specific B cells. Immunol Cell Biol, 81:163-170 (2003).
Kohler, G. & Milstein, C. Continuous cultures of fused cells secreting antibody of predefined specificity. Nature, 256:495-497 (1975).
Kukutsch N A, Rossner S, Austyn J M, Schuler G, Lutz M B. Formation and kinetics of MHC class I-ovalbumin peptide complexes on immature and mature murine dendritic cells. J Invest Dermatol 2000; 115(3):449-53.
Lev A, Denkberg G, Cohen C J, Tzukerman M, Skorecki K L, Chames P, et al. Isolation and characterization of human recombinant antibodies endowed with the antigen-specific, major histocompatibility complex-restricted specificity of T cells directed toward the widely expressed tumor T-cell epitopes of the telomerase catalytic subunit. Cancer Res 2002; 62(11):3184-94.
Liu, A. H., Creadon, G. & Wysocki, L. J. Sequencing heavy- and light-chain variable genes of single B-hybridoma cells by total enzymatic amplification. Proc Natl Acad Sci USA, 89:7610-7614 (1992).
Liu Z, Brattain M G, and Appert H. Links Differential display of reticulocalbin in the highly invasive cell line, MDA-MB-435, versus the poorly invasive cell line, MCF-7. Biochem Biophys Res Commun., 231(2):283-9 (1997).
Lou Y, Wang G, Lizee G, Kim G J, Finkelstein S E, Feng C, et al. Dendritic cells strongly boost the antitumor activity of adoptively transferred T cells in vivo. Cancer Res 2004; 64(18):6783-90.
Luescher, I. F. et al. HLA photoaffinity labeling reveals overlapping binding of homologous melanoma-associated gene peptides by HLA-A1, HLA-A29, and HLA-B44. J Biol Chem, 271:12463-12471 (1996).
Maeurer, M. J., Martin, D., Elder, E., Storkus, W. J. & Lotze, M. T. Detection of naturally processed and HLA-A1-presented melanoma T-cell epitopes defined by CD8(+) T-cells' release of granulocyte-macrophage colony-stimulating factor but not by cytolysis. Clin Cancer Res, 2:87-95 (1996).
Markovic S N, Suman V J, Ingle J N, Kaur J S, Pitot H C, Loprinzi C L, et al. Peptide vaccination of patients with metastatic melanoma: improved clinical outcome in patients demonstrating effective immunization. Am J Clin Oncol 2006; 29(4):352-60.
Menendez, J. A., R. Lupu, et al. Inhibition of tumor-associated fatty acid synthase hyperactivity induces synergistic chemosensitization of HER-2/neu-overexpressing human breast cancer cells to docetaxel (taxotere). Breast Cancer Res Treat, 84(2): 183-95 (2004a).
Menendez, J. A., S. Ropero, et al. Omega-6 polyunsaturated fatty acid gamma-linolenic acid (18:3n-6) enhances docetaxel (Taxotere) cytotoxicity in human breast carcinoma cells: Relationship to lipid peroxidation and HER-2/neu expression. Oncol Rep, 11(6): 1241-52 (2004b).
Messaoudi, I., J. LeMaoult, and J. Nikolic-Zugic, The mode of ligand recognition by two peptide:MHC class I-specific monoclonal antibodies. J Immunol, 163(6):3286-94 (1999).
Miki, T., Yano, S., Hanibuchi, M. & Sone, S. Bone metastasis model with multiorgan dissemination of human small-cell lung cancer (SBC-5) cells in natural killer cell-depleted SCID mice. Oncol Res, 12:209-217 (2000).
Morgan, R A, Dudley, M E, Yu, Y Y L, Zheng, Z, Robbins, R F, Theoret, M R, Wunderlich, J R, Hughes, M S, Restifo, N P, and Rosenberg, S A. J Immunol., 171:3287-3295 (2003).
Morley, S. J., et al. Differential requirements for caspase-8 activity in the mechanism of phosphorylation of eIF2α, cleavage of eIF4GI and signaling events associated with the inhibition of protein synthesis in apoptotic Jurkat T cells. FEBS Lett, 477(3): 229-36 (2000).
Morley, S. J., et al. Initiation factor modifications in the preapoptotic phase. Cell Death Differ, 12(6): 571-84 (2005).
Mosca P J, Hobeika A C, Clay T M, Morse M A, Lyerly H K. Direct detection of cellular immune responses to cancer vaccines. Surgery 2001; 129(3):248-54.
Murphy, D. B., et al., Monoclonal antibody detection of a major self peptide. MHC class II complex. J Immunol, 148(11): 3483-91 (1992).
Nahta, R., Hung, M. C. & Esteva, F. J. The HER-2-targeting antibodies trastuzumab and pertuzumab synergistically inhibit the survival of breast cancer cells. Cancer Res, 64:2343-2346 (2004).
Nakamura M, Iwahashi M, Nakamori M, Ueda K, Ojima T, Naka T, et al. Dendritic cells transduced with tumor-associated antigen gene elicit potent therapeutic antitumor immunity: comparison with immunodominant peptide-pulsed DCs. Oncology 2005; 68(⅔):163-70.
Nikitina, E. Y., et al. Dendritic cells transduced with full-length wild-type p53 generate antitumor cytotoxic T lymphocytes from peripheral blood of cancer patients. Clin Cancer Res, 7(1): 127-35 (2001).
Novellino L, Castelli C, Parmiani G. A listing of human tumor antigens recognized by T cells: March 2004 update. Cancer Immunol Immunother 2005; 54(3):187-207.
Oka Y, Tsuboi A, Kawakami M, Elisseeva O A, Nakajima H, Udaka K, et al. Development of WT1 peptide cancer vaccine against hematopoietic malignancies and solid cancers. Curr Med Chem 2006; 13(20):2345-52.
Oshiba, A., Renz, H., Yata, J. & Gelfand, E. W. Isolation and characterization of human antigen-specific B lymphocytes. Clin Immunol Immunopathol, 72:342-349 (1994).
Parker K C, Wiley D C. Overexpression of native human beta 2-microglobulin in Escherichia coli and its purification. Gene 1989; 83(1):117-24.
Parkhurst, M. R., et al., Immunization of patients with the hTERT:540-548 peptide induces peptide-reactive T lymphocytes that do not recognize tumors endogenously expressing telomerase. Clin Cancer Res, 10(14): 4688-98 (2004).
Petricciani J, Egan W, Vicari G, Furesz J, Schild G. Potency assays for therapeutic live whole cell cancer vaccines. Biologicals 2006.
Polakova, K., et al., Antibodies directed against the MHC-I molecule H-2Dd complexed with an antigenic peptide: similarities to a T cell receptor with the same specificity. J Immunol, 165(10): 5703-12 (2000).
Porgador A, Yewdell J W, Deng Y, Bennink J R, Germain R N. Localization, quantitation, and in situ detection of specific peptide-MHC class I complexes using a monoclonal antibody. Immunity 1997; 6(6):715-26.
Porgador, A., Yewdell, J. W., Deng, Y., Bennink, J. R. & Germain, R. N. Localization, quantitation, and in situ detection of specific peptide-MHC class I complexes using a monoclonal antibody. Immunity, 6:715-726 (1997).
Ramakrishna V, Treml J F, Vitale L, Connolly J E, O'Neill T, Smith P A, et al. Mannose receptor targeting of tumor antigen pmel17 to human dendritic cells directs anti-melanoma T cell responses via multiple HLA molecules. J Immunol 2004; 172(5):2845-52.
Reisbach, G., Gebhart, E. & Cailleau, R. Sister chromatid exchanges and proliferation kinetics of human metastatic breast tumor cells lines. Anticancer Res, 2:257-260 (1982).
Rognan, D., et al., Modeling the interactions of a peptide-major histocompatibility class I ligand with its receptors. I. Recognition by two alpha beta T cell receptors. J Comput Aided Mol Des, 14(1): 53-69 (2000).
Rongcun, Y. et al. Identification of new HER2/neu-derived peptide epitopes that can elicit specific CTL against autologous and allogeneic carcinomas and melanomas. J Immunol, 163:1037-1044 (1999).
Schlichtholz, B. et al. The immune response to p53 in breast cancer patients is directed against immunodominant epitopes unrelated to the mutational hot spot. Cancer Res, 52:6380-6384 (1992).
Seliger, B., et al. HER-2/neu is expressed in human renal cell carcinoma at heterogeneous levels independently of tumor grading and staging and can be recognized by HLA-A2.1-restricted cytotoxic T lymphocytes. Int J Cancer, 87(3): 349-59 (2000).
Shastri, N., Schwab, S. & Serwold, T. Producing nature's gene-chips: the generation of peptides for display by MHC class I molecules. Annu Rev Immunol, 20:463-493 (2002).
Slamon, D. J. et al. Use of chemotherapy plus a monoclonal antibody against HER2 for metastatic breast cancer that overexpresses HER2. N Engl J Med, 344:783-792 (2001).
Steenbakkers, P. G., et al., Localization of MHC class II/human cartilage glycoprotein-39 complexes in synovia of rheumatoid arthritis patients using complex-specific monoclonal antibodies. J Immunol, 170(11): 5719-27 (2003).
Steinman R M. Dendritic cells and immune-based therapies. Exp Hematol 1996; 24(8):859-62.
Sugita, Y, Wada, H, Fujita, S, Nakata, T, Sato, S, Noguchi, Y, et al. NY-ESO-1 expression and immunogeneity in malignant and benign breast tumors. Cancer Research, 64:2199-2204 (2004).
Sun, Y. et al. [Result of phase II clinical trial of herceptin in advanced Chinese breast cancer patients]. Zhonghua Zhong Liu Za Zhi, 25:581-583 (2003).
Theobald, M., et al. The sequence alteration associated with a mutational hotspot in p53 protects cells from lysis by cytotoxic T lymphocytes specific for a flanking peptide epitope. J Exp Med, 188(6):1017-28 (1998).
Theobald, M., et al. Targeting p53 as a general tumor antigen. Proc Natl Acad Sci USA, 92(26): 11993-7 (1995).
Townsend, S. E., Goodnow, C. C. & Cornall, R. J. Single epitope multiple staining to detect ultralow frequency B cells. J Immunol Methods, 249:137-146 (2001).
van der Burg, S. H., Visseren, M. J., Brandt, R. M., Kast, W. M. & Melief, C. J. Immunogenicity of peptides bound to MHC class I molecules depends on the MHC-peptide complex stability. J Immunol, 156: 3308-3314 (1996).
Vonderheide R H, Domchek S M, Schultze J L, George D J, Hoar K M, Chen D Y, Stephans K F, Masutomi K, Loda M, Xia Z, Anderson K S, Hahn W C, Nadler L M. Vaccination of cancer patients against telomerase induces functional antitumor CD8+ T lymphocytes. Clin Cancer Res., February 1; 10(3):828-39 (2004).
Wei M L, Cresswell P. HLA-A2 molecules in an antigen-processing mutant cell contain signal sequence-derived peptides. Nature 1992; 356(6368):443-6.
Weidanz J A, Nguyen T, Woodburn T, Neethling F A, Chiriva-Internati M, Hildebrand W H, et al. Levels of specific peptide-HLA class I complex predicts tumor cell susceptibility to CTL killing. J Immunol 2006; 177(8):5088-97.
Weidanz J A, Piazza P, Hickman-Miller H, Woodburn T, Nguyen T, Wahl A, et al. Development and implementation of a direct detection, quantitation and validation system for class I MHC self-peptide epitopes. J Immunol Methods 2007; 318:47-58.
Welch, W. R. et al. Antigenic heterogeneity in human ovarian cancer. Gynecol Oncol, 38:12-16 (1990).
Wherry E J, McElhaugh M J, Eisenlohr L C. Generation of CD8 (+) T cell memory in response to low, high, and excessive levels of epitope. J Immunol 2002; 168(9):4455-61.
Wherry E J, Puorro K A, Porgador A, Eisenlohr L C. The induction of virus-specific CTL as a function of increasing epitope expression: responses rise steadily until excessively high levels of epitope are attained. J Immunol 1999; 163(7):3735-45.
Whiteside T L, Gooding W. Immune monitoring of human gene therapy trials: potential application to leukemia and lymphoma. Blood Cells Mol Dis 2003; 31(1):63-71.
Whiteside T L, Zhao Y, Tsukishiro T, Elder E M, Gooding W, Baar J. Enzyme-linked immunospot, cytokine flow cytometry, and tetramers in the detection of T-cell responses to a dendritic cell-based multipeptide vaccine in patients with melanoma. Clin Cancer Res 2003; 9(2):641-9.
Wilkinson, K. A., Hudecz, F., Vordermeier, H. M., Ivanyi, J. & Wilkinson, R. J. Enhancement of the T cell response to a mycobacterial peptide by conjugation to synthetic branched polypeptide. Eur J Immunol, 29: 2788-2796 (1999).
Wittman V P, Woodburn D, Nguyen T, Neethling F A, Wright S, Weidanz J A. Antibody targeting to a class I MHC-peptide epitope promotes tumor cell death. J Immunol 2006; 177(6):4187-95.
Xia D, Moyana T, Xiang J. Combinational adenovirus-mediated gene therapy and dendritic cell vaccine in combating well established tumors. Cell Res 2006; 16(3):241-59.
Yang S, Linette G P, Longerich S, Haluska F G. Antimelanoma activity of CTL generated from peripheral blood mononuclear cells after stimulation with autologous dendritic cells pulsed with melanoma gp100 peptide G209-2M is correlated to TCR avidity. J Immunol 2002; 169(1):531-9.
Yang S, Linette G P, Longerich S, Roberts B L, Haluska F G. HLAA2.1/K (b) transgenic murine dendritic cells transduced with an adenovirus encoding human gp100 process the same A2.1-restricted peptide epitopes as human antigen-presenting cells and elicit A2.1-restricted peptide-specific CTL. Cell Immunol 2000; 204(1):29-37.
Yee C, Savage P A, Lee P P, Davis M M, Greenberg P D. Isolation of high avidity melanoma-reactive CTL from heterogeneous populations using peptide-MHC tetramers. J Immunol 1999; 162(4):2227-34.
Yu, Y. Y., Netuschil, N., Lybarger, L., Connolly, J. M. & Hansen, T. H. Cutting edge: single-chain trimers of MHC class I molecules form stable structures that potently stimulate antigen-specific T cells and B cells. J Immunol, 168: 3145-3149 (2002).
Yu, Z., et al. The use of transgenic mice to generate high affinity p53 specific cytolytic T cells. J Surg Res, 69(2): 33743 (1997).
Zehn D, Cohen C J, Reiter Y, Walden P. Efficiency of peptide presentation by dendritic cells compared with other cell types: implications for cross-priming. Int Immunol 2006.
Zehn D, Cohen C J, Reiter Y, Walden P. Extended presentation of specific MHC-peptide complexes by mature dendritic cells compared to other types of antigen-presenting cells. Eur J Immunol 2004; 34(6):1551-60.

1. A method of mediating lysis of tumorigenic cells expressing at least one specific peptide/MHC complex on a surface thereof, wherein the specific peptide of the at least one specific peptide/MHC complex is associated with a tumorigenic state, the method comprising the steps of:
providing a T cell receptor mimic comprising an antibody or antibody fragment reactive against a specific peptide/MHC complex, wherein the antibody or antibody fragment can differentiate the specific peptide/MHC complex from the MHC molecule alone, the specific peptide alone, and a complex of MHC and an irrelevant peptide, wherein the T cell receptor mimic is produced by immunizing a host with an effective amount of an immunogen comprising a multimer of two or more specific peptide/MHC complexes; and
contacting tumorigenic cells expressing at least one specific peptide/MHC complex on a surface thereof with the T cell receptor mimic, such that the T cell receptor mimic mediates lysis of the tumor cells expressing the at least one specific peptide/MHC complex on a surface thereof.

2. The method of claim 1, wherein the specific peptide is associated with at least one of breast cancer, ovarian cancer, prostate cancer, lung cancer, multiple myeloma, biliary cancer, and pancreatic cancer.

3. The method of claim 1, wherein the specific peptide is at least one of SEQ ID NOS:4, 5, 10, 18, 26, 29, 33 and 43.

4. The method of claim 1 wherein, in the step of contacting the tumorigenic cells expressing at least one specific peptide/MHC complex on a surface thereof with the T cell receptor mimic, the mechanism of mediation of cell lysis comprises activation of complement-dependent cytotoxicity (CDC).

5. The method of claim 1 wherein, in the step of contacting the tumorigenic cells expressing at least one specific peptide/MHC complex on a surface thereof with the T cell receptor mimic, the mechanism of mediation of cell lysis comprises activation of antibody-dependent cellular toxicity (ADCC).

6. The method of claim 1 wherein, in the step of contacting the tumorigenic cells expressing at least one specific peptide/MHC complex on a surface thereof with the T cell receptor mimic, the mechanism of mediation of cell lysis comprises induction of apoptosis.

7. The method of claim 1 wherein, in the step of contacting the tumorigenic cells expressing at least one specific peptide/MHC complex on a surface thereof with the T cell receptor mimic, the mechanism of mediation of cell lysis comprises activation of an anti-proliferative effect.

8. The method of claim 1 wherein, in the step of providing a T cell receptor mimic, the T cell receptor mimic has a binding affinity of about 10 nanomolar or greater.

9. A method of mediating lysis of infected cells expressing at least one specific peptide/MHC complex on a surface thereof, wherein the specific peptide of the at least one specific peptide/MHC complex is associated with an infectious disease state, the method comprising the steps of:
providing a T cell receptor mimic comprising an antibody or antibody fragment reactive against a specific peptide/MHC complex, wherein the antibody or antibody fragment can differentiate the specific peptide/MHC complex from the MHC molecule alone, the specific peptide alone, and a complex of MHC and an irrelevant peptide, wherein the T cell receptor mimic is produced by immunizing a host with an effective amount of an immunogen comprising a multimer of two or more specific peptide/MHC complexes; and
contacting infected cells expressing at least one specific peptide/MHC complex on a surface thereof with the T cell receptor mimic, such that the T cell receptor mimic mediates lysis of the infected cells expressing the at least one specific peptide/MHC complex on a surface thereof.

10. The method of claim 9, wherein the specific peptide is associated with a bacterial infection.

11. The method of claim 9, wherein the specific peptide is associated with a viral infection.

12. The method of claim 11, wherein the specific peptide is associated with HIV infection.

13. The method of claim 12, wherein the specific peptide is at least one of SEQ ID NOS:34 and 73.

14. The method of claim 11, wherein the specific peptide is associated with infection with a flavivirus.

15. The method of claim 14, wherein the specific peptide is associated with infection with West Nile virus.

16. The method of claim 15, wherein the specific peptide is at least one of SEQ ID NOS:63 and 78-82.

17. The method of claim 11, wherein the specific peptide is associated with infection with influenza virus.

18. The method of claim 17, wherein the specific peptide is at least one of SEQ ID NOS:1, 28, 37, 50, 53, 54 and 83-88.

19. The method of claim 11, wherein the specific peptide is associated with infection with at least one virus selected from the group consisting of hepatitis B, hepatitis C, human papilloma virus (HPV), herpes virus, cytomegalovirus (CMV) and Epstein-Barr virus (EBV).

20. The method of claim 9 wherein, in the step of contacting the infected cells expressing at least one specific peptide/MHC complex on a surface thereof with the T cell receptor mimic, the mechanism of mediation of cell lysis comprises activation of complement-dependent cytotoxicity (CDC).

21. The method of claim 9 wherein, in the step of contacting the infected cells expressing at least one specific peptide/MHC complex on a surface thereof with the T cell receptor mimic, the mechanism of mediation of cell lysis comprises activation of antibody-dependent cellular toxicity (ADCC).

22. The method of claim 9 wherein, in the step of contacting the infected cells expressing at least one specific peptide/MHC complex on a surface thereof with the T cell receptor mimic, the mechanism of mediation of cell lysis comprises induction of apoptosis.

23. The method of claim 9 wherein, in the step of contacting the infected cells expressing at least one specific peptide/MHC complex on a surface thereof with the T cell receptor mimic, the mechanism of mediation of cell lysis comprises activation of an anti-proliferative effect.

24. The method of claim 9 wherein, in the step of providing a T cell receptor mimic, the T cell receptor mimic has a binding affinity of about 10 nanomolar or greater.

25. A method of blocking autoreactive T cells activated by cells expressing at least one specific peptide/MHC complex on a surface thereof, wherein the specific peptide of the at least one specific peptide/MHC complex is associated with an autoimmune state, the method comprising the steps of:
providing a T cell receptor mimic comprising an antibody or antibody fragment reactive against a specific peptide/MHC complex, wherein the antibody or antibody fragment can differentiate the specific peptide/MHC complex from the MHC molecule alone, the specific peptide alone, and a complex of MHC and an irrelevant peptide, wherein the T cell receptor mimic is produced by immunizing a host with an effective amount of an immunogen comprising a multimer of two or more specific peptide/MHC complexes; and
contacting cells expressing at least one specific peptide/MHC complex on a surface thereof with the T cell receptor mimic, such that the T cell receptor mimic binds to the surface of the cell and blocks binding and activation of autoreactive T cells.

26. The method of claim 25, wherein the specific peptide is at least one of SEQ ID NOS:2 and 4.

27.-52. (canceled)

53. A method of killing or damaging a target cell expressing or displaying an antigen-presenting portion of a complex composed of a human antigen-presenting molecule and an antigen derived from a pathogen, the method comprising:
exposing the target cell to a T cell receptor mimic comprising an antibody or antibody fragment reactive against a specific peptide/MHC complex, wherein the antibody or antibody fragment can differentiate the specific peptide/MHC complex from the MHC molecule alone, the specific peptide alone, and a complex of MHC and an irrelevant peptide, wherein the T cell receptor mimic is produced by immunizing a host with an effective amount of an immunogen comprising a multimer of two or more specific peptide/MHC complexes, thereby killing or damaging a target cell expressing or displaying an antigen-presenting portion of a complex composed of a human antigen-presenting molecule and an antigen derived from a pathogen.


SEQUENCE LISTING (97 sequences)

SEQ ID NO: 1 - protein, 7 aa, Influenza virus
AEAMEVA

SEQ ID NO: 2 - protein, 9 aa, Homo sapiens
TMTRVLQGV

SEQ ID NO: 3 - protein, 9 aa, Homo sapiens
VLQGVLPAL

SEQ ID NO: 4 - protein, 9 aa, Homo sapiens
GVLPALPQV

SEQ ID NO: 5 - protein, 9 aa, Homo sapiens
KIFGSLAFL

SEQ ID NO: 6 - protein, 9 aa, Homo sapiens
EVDPIGHLY

SEQ ID NO: 7 - protein, 10 aa, Homo sapiens
GPRTAALGLL

SEQ ID NO: 8 - protein, 9 aa, Homo sapiens
LLGRNSFEV

SEQ ID NO: 9 - protein, 9 aa, Homo sapiens
VLMTEDIKL

SEQ ID NO: 10 - protein, 9 aa, Homo sapiens
YLLPAIVHI

SEQ ID NO: 11 - protein, 9 aa, Homo sapiens
TLAYLIFCL

SEQ ID NO: 12 - protein, 8 aa, Homo sapiens
YLEPGPVT

SEQ ID NO: 13 - protein, 9 aa, Homo sapiens
SLLMWITQV

SEQ ID NO: 14 - protein, 9 aa, Homo sapiens
ILAKFLHWL

SEQ ID NO: 15 - protein, 9 aa, Homo sapiens
AAGIGILTV

SEQ ID NO: 16 - protein, 9 aa, Homo sapiens
AIMDKNIIL

SEQ ID NO: 17 - protein, 9 aa, Homo sapiens
ALGIGILTV

SEQ ID NO: 18 - protein, 9 aa, Homo sapiens
ALMPVLNQV

SEQ ID NO: 19 - protein, 9 aa, Homo sapiens
ATDFKFAMY

SEQ ID NO: 20 - protein, 9 aa, Homo sapiens
ATTNILEHY

SEQ ID NO: 21 - protein, 9 aa, Homo sapiens
AVLPPLPQV

SEQ ID NO: 22 - protein, 9 aa, Homo sapiens
EADPTGHSY

SEQ ID NO: 23 - protein, 10 aa, Homo sapiens
ELTLGEFLKL

SEQ ID NO: 24 - protein, 11 aa, Homo sapiens
FLAEDALIITV

SEQ ID NO: 25 - protein, 10 aa, Homo sapiens
FLSTLTIDGV

SEQ ID NO: 26 - protein, 9 aa, Homo sapiens
FLSELTQQL

SEQ ID NO: 27 - protein, 9 aa, Homo sapiens
FLYDDNQRV

SEQ ID NO: 28 - protein, 9 aa, Influenza virus
GILGFVFTL

SEQ ID NO: 29 - protein, 9 aa, Homo sapiens
GLNEEIARV

SEQ ID NO: 30 - protein, 9 aa, Homo sapiens
GVLPNIQAV

SEQ ID NO: 31 - protein, 10 aa, Homo sapiens
GVYDGEEHSV

SEQ ID NO: 32 - protein, 9 aa, Homo sapiens
IADMGHLKY

SEQ ID NO: 33 - protein, 9 aa, Homo sapiens
ILDQKINEV

SEQ ID NO: 34 - protein, 9 aa, HIV
ILKEPVHGV

SEQ ID NO: 35 - protein, 9 aa, Homo sapiens
ILNSRPPSV

SEQ ID NO: 36 - protein, 9 aa, Homo sapiens
IMDQVPFSV

SEQ ID NO: 37 - protein, 9 aa, Influenza virus
IPSIQSRGL

SEQ ID NO: 38 - protein, 9 aa, Homo sapiens
ITDQVPFSV

SEQ ID NO: 39 - protein, 9 aa, Homo sapiens
ITNSRPPSV

SEQ ID NO: 40 - protein, 9 aa, Homo sapiens
KIFGALAFL

SEQ ID NO: 41 - protein, 9 aa, Homo sapiens
KIFGGLAFL

SEQ ID NO: 42 - protein, 9 aa, Homo sapiens
KIFGKLAFL

SEQ ID NO: 43 - protein, 9 aa, Homo sapiens
KIGEGTYGV

SEQ ID NO: 44 - protein, 16 aa, Homo sapiens
KKLLTQHFVQENYLEY

SEQ ID NO: 45 - protein, 9 aa, Homo sapiens
KLGEGTYGV

SEQ ID NO: 46 - protein, 9 aa, Homo sapiens
KLMSPKLYV

SEQ ID NO: 47 - protein, 9 aa, Homo sapiens
KLQELNYNL

SEQ ID NO: 48 - protein, 9 aa, Homo sapiens
KVLEYVIKV

SEQ ID NO: 49 - protein, 9 aa, Homo sapiens
LKMESLNFI

SEQ ID NO: 50 - protein, 9 aa, Influenza
LPFDRTTVM

SEQ ID NO: 51 - protein, 9 aa, RSV
NAITNAKII

SEQ ID NO: 52 - protein, 9 aa, CMV
NLVPMVATV

SEQ ID NO: 53 - protein, 9 aa, Influenza virus
QPEWFRNIL

SEQ ID NO: 54 - protein, 9 aa, Influenza
QPEWFRNVL

SEQ ID NO: 55 - protein, 9 aa, Homo sapiens
RMFPNAPYL

SEQ ID NO: 56 - protein, 9 aa, Homo sapiens
RPYSNVSNL

SEQ ID NO: 57 - protein, 9 aa, Dengue virus
SIGGVFTSV

SEQ ID NO: 58 - protein, 9 aa, Homo sapiens
SLFLGILSV

SEQ ID NO: 59 - protein, 9 aa, Homo sapiens
SLLMWITQC

SEQ ID NO: 60 - protein, 9 aa, Homo sapiens
SLLEKREKT

SEQ ID NO: 61 - protein, 9 aa, Homo sapiens
STAPPAHGV

SEQ ID NO: 62 - protein, 9 aa, Homo sapiens
STPPPGTRV

SEQ ID NO: 63 - protein, 9 aa, West Nile Virus
SVGGVFTSV

SEQ ID NO: 64 - protein, 9 aa, HRSV
SYIGSINNI

SEQ ID NO: 65 - protein, 9 aa, HPV
TLHEYMLDL

SEQ ID NO: 66 - protein, 9 aa, HPV
TLQDIVLHL

SEQ ID NO: 67 - protein, 9 aa, Homo sapiens
TMMRVLQAV

SEQ ID NO: 68 - protein, 9 aa, Homo sapiens
TPQSNRPVM

SEQ ID NO: 69 - protein, 9 aa, Homo sapiens
VLQAVLPPL

SEQ ID NO: 70 - protein, 9 aa, Homo sapiens
VLQELNVTV

SEQ ID NO: 71 - protein, 10 aa, Homo sapiens
VMAGVGSPYV

SEQ ID NO: 72 - protein, 9 aa, Homo sapiens
YIFGSLAFL

SEQ ID NO: 73 - protein, 13 aa, HIV
YKYKVVKIEPLGV

SEQ ID NO: 74 - protein, 9 aa, Homo sapiens
YLEPGPVTA

SEQ ID NO: 75 - protein, 9 aa, Homo sapiens
YLEPGPVTV

SEQ ID NO: 76 - protein, 9 aa, Epstein-Barr virus
YLLEMLWRL

SEQ ID NO: 77 - protein, 10 aa, Homo sapiens
YMLDLQPETT

SEQ ID NO: 78 - protein, 10 aa, West Nile Virus
RLDDDGNFQL

SEQ ID NO: 79 - protein, 9 aa, West Nile Virus
ATWAENIQV

SEQ ID NO: 80 - protein, 9 aa, West Nile Virus
YTMDGEYRL

SEQ ID NO: 81 - protein, 9 aa, West Nile Virus
SLTSINVQA

SEQ ID NO: 82 - protein, 9 aa, West Nile Virus
SLFGQRIEV

SEQ ID NO: 83 - protein, 8 aa, Influenza virus
AAEAMEVA

SEQ ID NO: 84 - protein, 10 aa, Influenza virus
LKNDLLENLQ

SEQ ID NO: 85 - protein, 9 aa, Influenza virus
LPFDKTTIM

SEQ ID NO: 86 - protein, 9 aa, Influenza virus
LPFEKSTIM

SEQ ID NO: 87 - protein, 9 aa, Influenza
SPIVPSFDM

SEQ ID NO: 88 - protein, 9 aa, Influenza virus
NPIVPSFDM

SEQ ID NO: 89 - protein, 9 aa, Dengue virus
SLGGVFTSI

SEQ ID NO: 90 - protein, 9 aa, Dengue virus
SVGGVLNSL

SEQ ID NO: 91 - protein, 9 aa, Dengue virus
SVGGLFTSL

SEQ ID NO: 92 - protein, 9 aa, JEV/SEV
SIGGVFNSI

SEQ ID NO: 93 - protein, 9 aa, Murray Valley Encephalitis virus
SVGGVFNSI

SEQ ID NO: 94 - protein, 9 aa, Yellow Fever Virus
SAGGFFTSV

SEQ ID NO: 95 - protein, 10 aa, CMV
NLVPMVVATV

SEQ ID NO: 96 - protein, 9 aa, Homo sapiens
SLLMWIQTV

SEQ ID NO: 97 - protein, 9 aa, Homo sapiens
YLEVGPVTA